(19) United States
(12) Patent Application Publication
(10) Pub. No.: US 2026/0327874 A1
(43) Pub. Date: Oct. 22, 2026

(54) Formation Surface with a Reference Line and Method for Manufacturing Zoned Webs

(71) Applicant: Kimberly-Clark Worldwide, Inc., Neenah, WI (US)

(72) Inventors: James L. Cahall, Appleton, WI (US); Antonio J. Carrillo Ojeda, Appleton, WI (US); Steven J. Roffers, Neenah, WI (US); Dustin J. Smith, Green Bay, WI (US); Folau Piula Penaia, Altona, Victoria (AU); Teresa Kubicki, Greensborough, Victoria (AU)

(21) Appl. No.: 19/108,631

(22) PCT Filed: Sep. 21, 2022
(86) PCT No.: PCT/US2022/044253

§ 371 Date: Mar. 4, 2025

(51) Int. Cl. D04H 1/495; D04H 1/498; D04H 1/74; D04H 18/04

(52) U.S. Cl. CPC D04H 1/495; D04H 1/498; D04H 1/74; D04H 18/04; D10B 2509/026

(57) ABSTRACT

A formation surface includes a wall that defines a plurality of lanes with discrete feature zones. The wall further includes a reference line with a plurality of features. The formation surface may be used to modify a substrate to include a reference line index. The reference line index may be used to register the substrate during slitting of the substrate.

### BACKGROUND

Nonwoven materials are frequently used within personal care absorbent articles, such as diapers or incontinence briefs. Moreover, an inner lining of such articles face and contact the skin of a wearer. Contact between body exudates, such as semi solid fecal material, captured within the articles and the skin of the wearer can cause discomfort. Moving body exudates through the inner lining and away from the skin of the wearer can reduce or limit such discomfort.

Nonwoven materials are subjected various manufacturing processes to form personal care absorbent articles with the nonwoven materials. For instance, a roll of nonwoven material can be unwound and cut to form a layer in such articles. Registration can assist with accurate cutting of the nonwoven material from the roll. Certain registration systems measure light passing through the nonwoven material. However, such registration is affected by apertures within the nonwoven material. Thus, a nonwoven material with features that facilitate registration of the nonwoven material would be useful.

### SUMMARY

In general, the present disclosure is directed to a formation surface with a reference line, such as a plurality of features on a side of the formation surface. The formation surface may be used to form a web with a plurality of apertures corresponding to the reference line. Apertures formed by the reference line can be used to register cutting of the web. The formation surface is particularly useful for slitting a web with a plurality of lanes each having discrete aperture zones. Gaps between the discrete aperture zones can hinder registration using the discrete aperture zones. In contrast, the apertures formed by the reference line can provide a more consistent reference index relative to the discrete aperture zones. The web manufactured with the formation surface may be incorporated within an absorbent article, such as a pad, diaper, disposable undergarment, etc.

In one example embodiment, a formation surface, includes a drum with a plurality of lanes that are spaced apart along an axial direction on the drum. The plurality of lanes includes a first lane and a second lane. The first lane includes one or more discrete feature zones. The second lane includes one or more discrete feature zones. The drum further includes a reference line. The reference line includes a plurality of features that are distributed around the drum along the circumferential direction. The features of the reference line are spaced from the first lane along the axial direction.

In another example embodiment, a formation surface includes a wall with a plurality of lanes that are spaced apart along a lateral direction on the wall. The plurality of lanes includes a first lane and a second lane. The first lane includes one or more discrete feature zones that are spaced apart along a longitudinal direction on the wall. The second lane includes one or more discrete feature zones that are spaced apart along the longitudinal direction on the wall. The wall further includes a reference line. The reference line includes a plurality of features that are distributed on the wall along the longitudinal direction. The features of the reference line are spaced from the first lane along the lateral direction.

In another example embodiment, a nonwoven material, includes a plurality of fibers forming a nonwoven fibrous web defining a lateral direction and a longitudinal direction. The lateral and longitudinal directions are perpendicular. The nonwoven fibrous web includes a plurality of discrete feature zone lanes and a reference line. The plurality of discrete feature zone lanes are spaced apart along the lateral direction. The plurality of discrete feature zone lanes includes a first lane and a second lane. The first lane includes one or more discrete feature zones, and the second lane includes one or more discrete feature zones. The reference line includes a plurality of features that are distributed along the longitudinal direction. The features of the reference line are spaced from the first lane along the lateral direction.

These and other features, aspects and advantages of the present disclosure will become better understood with reference to the following description and appended claims. The accompanying drawings, which are incorporated in and constitute a part of this specification, illustrate embodiments of the disclosure and, together with the description, serve to explain the principles of the disclosure.

### BRIEF DESCRIPTION OF THE DRAWINGS

A full and enabling disclosure of the present disclosure, including the best mode thereof, directed to one of ordinary skill in the art, is set forth in the specification, which makes reference to the appended figures.

FIG. 1 is a process schematic depicting a manufacturing method including forming zoned webs, according to example aspects of the present disclosure.

FIG. 2 is a top plan view of a surface of a pattern roll, laid flat, such as from the pattern roll used in the apparatus of FIG. 1.

FIG. 3 is a partial, top plan view of a drum with a formation surface, such as may be used in the apparatus of FIG. 1.

FIG. 4 is a detailed view of a first pattern zone of the pattern roll taken from FIG. 2.

FIG. 5 is a detailed view of a second pattern zone of the pattern roll taken from FIG. 2.

FIG. 6 is a top plan view of a portion of a modified substrate formable with the apparatus of FIG. 1, and which includes three lanes with a plurality of first discrete aperture zones and a plurality of second discrete aperture zones.

FIG. 7 is a top, plan view of a nonwoven material according to an example embodiment of the present disclosure.

FIG. 8 is an image providing a detailed view of nodes of the example nonwoven material of FIG. 1.

FIG. 9 is a perspective view of example equipment and set-up to perform the Material Sample Analysis Test Method as described herein.

Repeat use of reference characters in the present specification and drawings is intended to represent the same or analogous features or elements of the present invention.

### DETAILED DESCRIPTION

In an embodiment, the present disclosure is generally directed towards a formation surface with features for improved registration of a nonwoven substrate modified by the formation surface. It is to be understood by one of ordinary skill in the art that the present discussion is a description of exemplary embodiments only, and is not intended as limiting the broader aspects of the present disclosure.

When introducing elements of the present disclosure or the preferred embodiment(s) thereof, the articles “a”, “an”, “the” and “said” are intended to mean that there are one or more of the elements. As used herein, the terms “includes” and “including” are intended to be inclusive in a manner similar to the term “comprising.” Similarly, the term “or” is generally intended to be inclusive (i.e., “A or B” is intended to mean “A or B or both”). Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. For example, the approximating language may refer to being within a ten percent (10%) margin.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “fluid entangling”, and “fluid-entangled” generally refers herein to a formation process for further increasing the degree of fiber entanglement within a given fibrous nonwoven web or between fibrous nonwoven webs and other materials so as to make the separation of the individual fibers and/or the layers more difficult as a result of the entanglement. Generally, this is accomplished by supporting the fibrous nonwoven web on some type of forming or carrier surface which has at least some degree of permeability to the impinging pressurized fluid. A pressurized fluid stream (usually multiple streams) is then directed against the surface of the nonwoven web which is opposite the supported surface of the web. The pressurized fluid contacts the fibers and forces portions of the fibers in the direction of the fluid flow thus displacing all or a portion of a plurality of the fibers towards the supported surface of the web. The result is a further entanglement of the fibers in what can be termed the Z-direction of the web (the thickness) relative to its more planar dimension, the X-Y plane. When two or more separate webs or other layers are placed adjacent one another on the forming/carrier surface and subjected to the pressurized fluid, the generally desired result is that some of the fibers of at least one of the webs are forced into the adjacent web or layer thereby causing fiber entanglement between the interfaces of the two surfaces so as to result in the bonding or joining of the webs/layers together due to the increased entanglement of the fibers. The degree of bonding or entanglement will depend on a number of factors including, but not limited to, the types of fibers being used, the fiber lengths, the degree of pre-bonding or entanglement of the web or webs prior to subjection to the fluid entangling process, the type of fluid being used (liquids, such as water, steam or gases, such as air), the pressure of the fluid, the number of fluid streams, the speed of the process, the dwell time of the fluid and the porosity of the web or webs/other layers and the forming/carrier surface. One of the most common fluid entangling processes is referred to as hydroentangling, which is a well-known process to those of ordinary skill in the art of nonwoven webs. Examples of fluid entangling process can be found in U.S. Pat. No. 4,939,016 to Radwanski et al., U.S. Pat. No. 3,485,706 to Evans, and U.S. Pat. Nos. 4,970,104 and 4,959,531 to Radwanski, each of which is incorporated herein in its entirety by reference thereto for all purposes.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Forming Method and Apparatus:

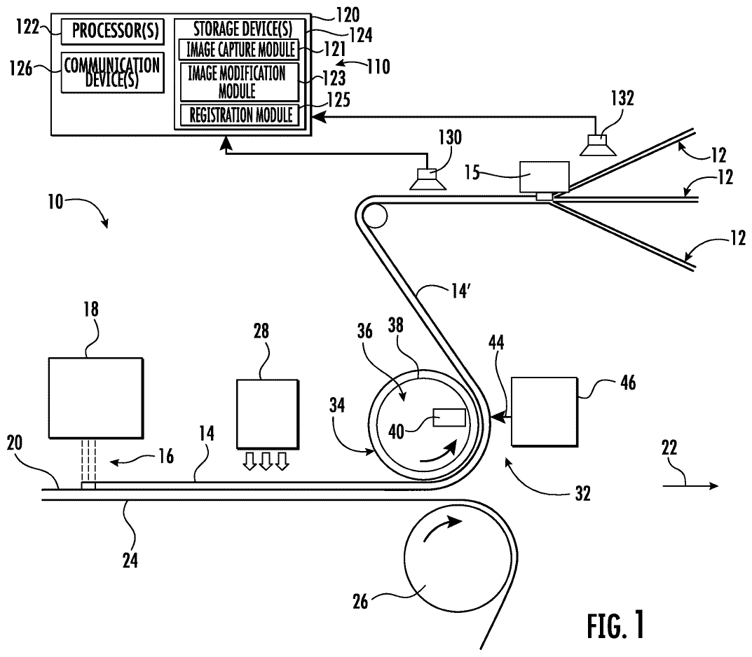

An example method and apparatus 10 for manufacturing zoned webs 12 is depicted in FIG. 1. As will be described herein, method 10 for manufacturing zoned webs 12 may include a fluid entanglement process, however, it can be appreciated that other techniques for modifying a substrate 14 in order to provide zoned webs 12 and are within the scope of example aspects of the disclosure. As illustrated in FIG. 1, the method 10 may include providing a substrate 14. The substrate 14 may include a plurality of fibers. The substrate 14 may be formed from a variety of techniques of web forming, such as, but not limited to a wet-laying, a foam-laying, or a carding process. In the example embodiment depicted in FIG. 1, the substrate 14 may be formed by a wet-laying process through a fiber and water slurry 16 being deposited from a drum 18 on a precursor forming surface 20. The precursor forming surface 20 as shown in FIG. 1 may be a precursor material, such as a spunbond web. However, it is contemplated that the fiber and water slurry 16 may be deposited directly on a belt, screen, or other surface that provides a precursory forming surface 20.

Where the precursor forming surface 20 is a precursor material, example suitable materials include spunbond materials, bonded-carded materials (for example, such as through air bonded-carded webs (TABCW)), spunlace materials, and any laminates of such materials—including spunbonded-meltblown-spunbond (SMS) webs and similar classes of materials. Preferable basis weights for such precursor materials include materials having basis weights of between about 10 gsm and about 80 gsm, such as between about 10 gsm and about 50 gsm, such as between about 10 gsm and about 30 gsm. Although, other basis weights are contemplated, depending on an end use of the final material. Fibers of the precursor web material may be any suitable fiber type, including being formed of polyolefin materials as is typical for such nonwoven webs in the art. Further, the fibers may have deniers of between about 0.5 and 5.0, such as between about 0.7 and 4.0, such as between about 0.9 and 3.0, such as between about 1.0 and about 2.0. The fibers may have typical staple fiber lengths of less than one hundred millimeters (100 mm). In some example embodiments, the fibers of the precursor web may be similar to those described with respect to the support web 14 in U.S. Pat. No. 9,327,473, titled “Fluid-entangled laminate webs having hollow projections and a process and apparatus for making the same”, to Scott Kirby et al., the entirety of which is hereby incorporated by reference.

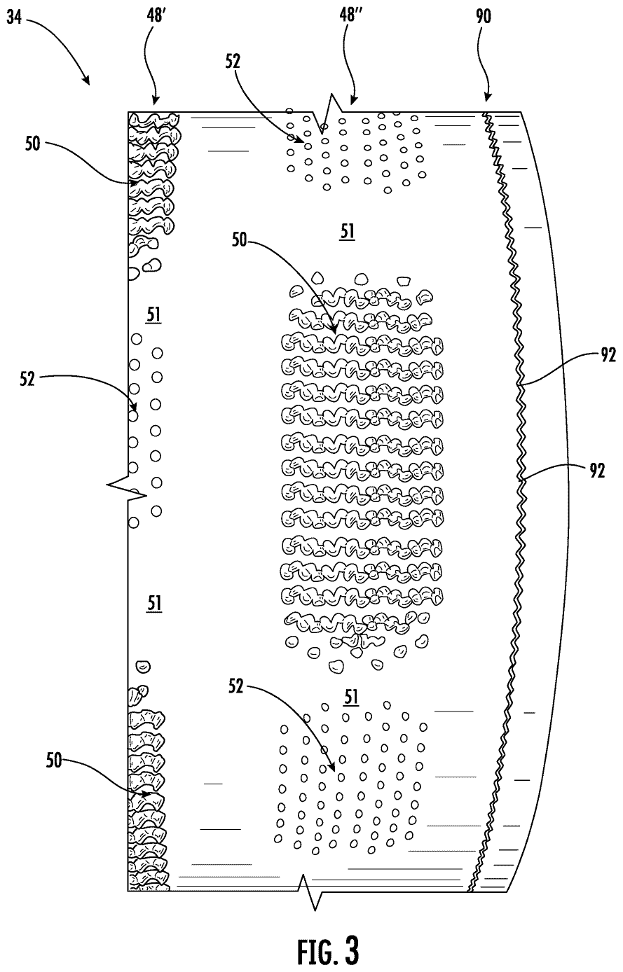

In further example embodiments according to the present disclosure, the precursor forming surface 20 may not be a precursor web, and the method 10 may further include combining a support web with the substrate 14 with fluid entanglement device 46—whose function is described in more detail below. In such example embodiments, the support web may be similar in terms of fiber content and construction to the above-described precursor web. Such a method is described in more detail with respect to FIG. 3 of U.S. Pat. No. 9,327,473 to Scott Kirby et al.

As mentioned, the substrate 14 may be formed according to a variety of techniques (e.g. wet-laying, a foam-laying, or a carding process). The substrate 14 may preferably have a basis weight of between about 10 gsm and about 80 gsm, such as between about 20 gsm and about 70 gsm, such as between about 30 gsm and about 60 gsm. The substrate 14 may be formed of typical polyolefin fibers or any suitable polyester-class fibers. Such fibers may preferably have deniers of between about 0.5 and 5.0, such as between about 0.7 and 4.0, such as between about 0.9 and 3.0, such as between about 1.0 and about 2.0. The fibers may have typical staple fiber lengths of less than one hundred millimeters (100 mm). In some example embodiments, the fibers of the substrate 14 may be similar to those described with respect to the projection web 16 in U.S. Pat. No. 9,327,473 to Scott Kirby et al.

The substrate 14 may be transferred in a machine direction by a belt 24 driven by a drive roll 26, or other transfer devices known by one of ordinary skill in the art. If the substrate 14 is formed through a wet-laying process, the substrate 14 may be dried through known techniques with a dryer 28.

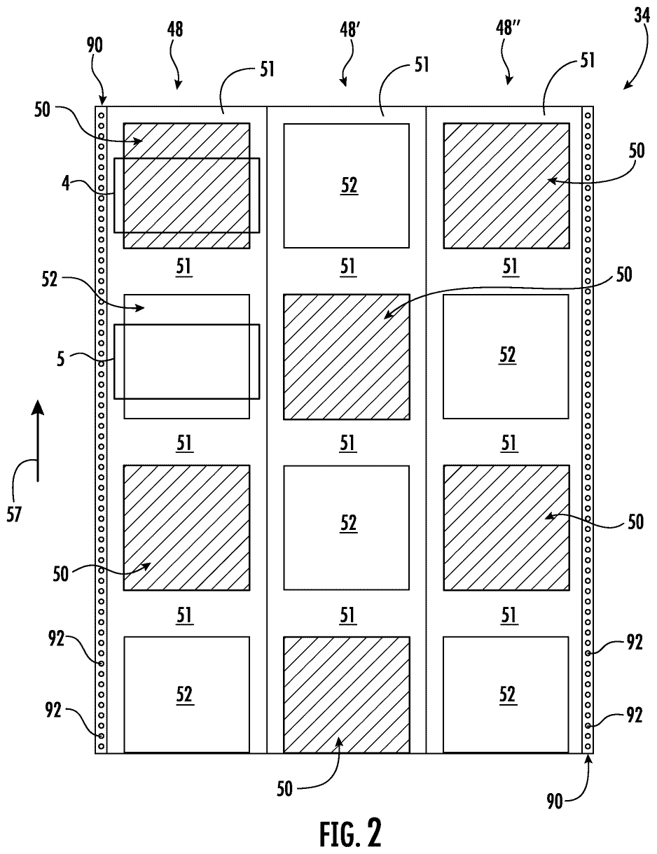

In an example embodiment that includes fluid entanglement, the method may include modifying the substrate 14 with a fluid entanglement apparatus 32. Whether completed off-line or in-line, the substrate 14 may be transferred to a pattern wall or surface 34. The pattern surface 34 may be a surface of a texturizing drum 36, such as a forming screen. A portion of an exemplary pattern surface 34 will be described in greater detail below, and are shown in FIGS. 2, 3, 4, and 5. The texturizing drum 36 may rotate as shown in FIG. 1 and may be driven by any suitable drive mechanism (not shown), such as electric motors and gearing as are well known to those of ordinary skill in the art. The material forming the texturizing drum 36 may be any number of suitable materials commonly used for such forming drums including, but not limited to, sheet metal, plastics and other polymer materials, rubber, etc. The pattern surface 34 may be perforated to allow for fluid to pass through the texturizing drum 36 and modify the substrate 14 through fluid entanglement. In example embodiments, the pattern surface 34 may be removably fitted onto and over an optional porous inner drum shell 38 so that different pattern surfaces 34 may be used for different end product designs.

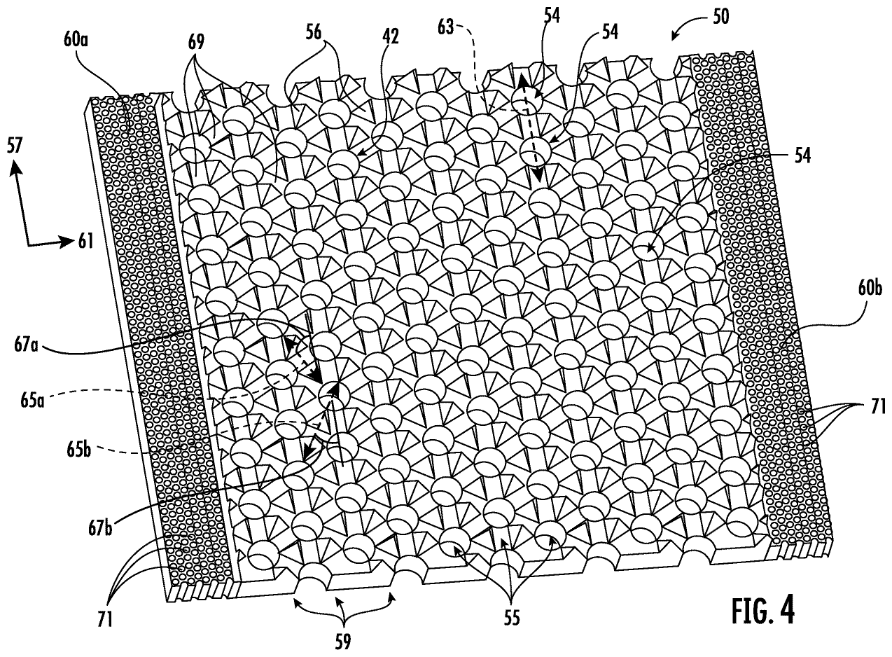

The porous inner drum shell 38 may interface with a fluid removal system 40, which facilitates pulling the entangling fluid and through the pattern surface 34, and in some patterns of the pattern surface 34, also facilitates pulling fibers of the substrate 14 through the pattern surface 34, to form three dimensional structures on the modified substrate 14′, if desired. The porous inner drum shell 38 may also act as a barrier to retard further fiber movement down into the fluid removal system 40 and other portions of the equipment thereby reducing fouling of the equipment. The porous inner drum shell 38 may rotate in the same direction and at the same speed as the texturizing drum 36. In addition, to further control the height of any three dimensional features on the modified substrate 14′, the distance between the inner drum shell 38 and the base plane 42 (labeled in FIG. 4) of the pattern surface 34 may be varied. In example embodiments, the spacing between the base plane 42 of the pattern surface 34 and the outer surface of the inner drum shell 38 may range between about zero millimeters (0 mm) and about five millimeters (5 mm). Other ranges can be used depending on the particular end-use application and the desired features of the modified substrate 14′ which will ultimately form the zoned webs 12.

The fluid entanglement apparatus 32 may include a plurality of high pressure fluid jets (not shown) to emit a plurality of pressurized fluid streams 44 from a fluid entanglement device 46. In some example embodiments, there may be more than one fluid entanglement device 46. The most common fluid used in this regard is referred to as spunlace or hydroentangling technology, which uses pressurized water as the fluid for entanglement. These fluid streams 44, which are preferably water, may be directed towards the substrate 14 on the pattern surface 34 and can cause the fibers to be further entangled within substrate 14 and/or the precursor forming surface 20 (in the case the precursor forming surface is an underlying web of material). As will be described in more detail below, the fluid streams 44 can modify the substrate 14 to provide a modified substrate 14′.

The entangling fluid streams 44 of the fluid entangling devices 46 may emanate from injectors via jet packs or strips (not shown) that include a row or rows of pressurized fluid jets with small apertures of a diameter usually between eight-hundredths of a millimeter (0.08 mm) and fifteen-hundredths of a millimeter (0.15 mm) and spacing of around a half millimeter (0.5 mm) in the cross-machine direction. The pressure in the jets may be between about five (5) bar and about four hundred (400) bar but typically is less than two hundred (200) bar except for heavy nonwoven materials and when fibrillation is required. Other jet sizes, spacings, numbers of jets and jet pressures may be used depending upon the particular end application. Such fluid entangling devices 66 are well known to those of ordinary skill in the art and are readily available from such manufacturers as Fleissner of Germany and Andritz-Perfojet of France.

The fluid entangling devices 46 may have the jet orifices positioned or spaced between about five millimeters (5 mm) and about twenty millimeters (20 mm), and more typically between about five millimeters (5 mm) and about ten millimeters (10 mm) from the pattern surface 34 though the actual spacing may vary depending on the basis weights of the materials being acted upon, the fluid pressure, the number of individual jets being used, the amount of vacuum being used via the fluid removal system 40, and the speed at which the equipment is being run.

In the example embodiment shown in FIG. 1, the fluid entangling device 46 may a conventional hydroentangling device the construction and operation of which is well known to those of ordinary skill in the art, such as, for example, U.S. Pat. No. 3,485,706 to Evans, the contents of which is incorporated herein by reference in its entirety for all purposes. Also see the description of the hydraulic entanglement equipment described by Honeycomb Systems, Inc., Biddeford, Me., in the article entitled “Rotary Hydraulic Entanglement of Nonwovens”, reprinted from INSIGHT '86 INTERNATIONAL ADVANCED FORMING/BONDING Conference, the contents of which is incorporated herein by reference in its entirety for all purposes.

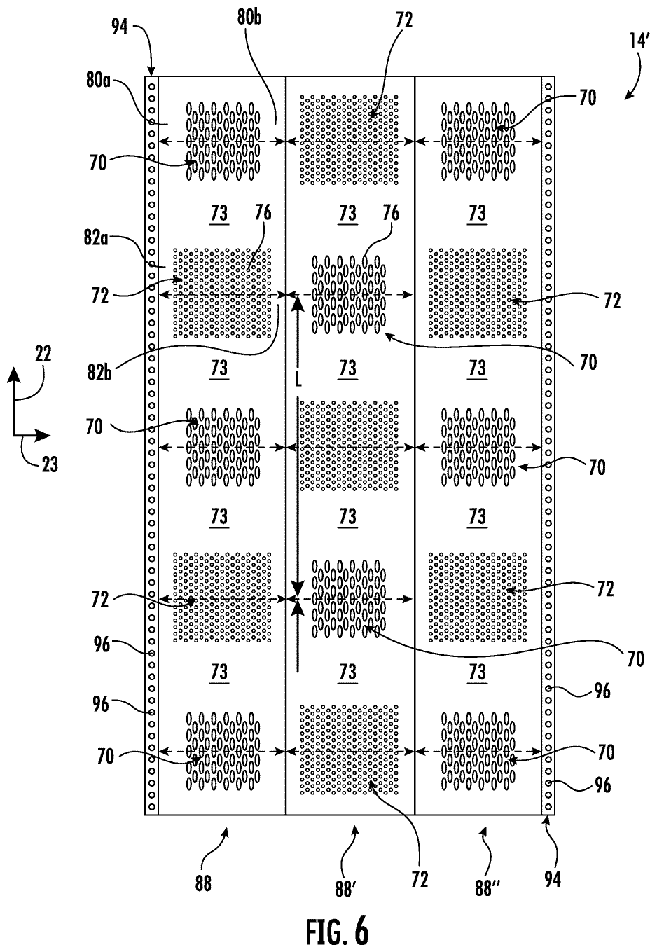

While described in the context of fluid entangling with the fluid entangling device 46, it will be understood that features of the modified substrate 14′, such as nodes 74, apertures 76, and connecting ligaments 78 (FIG. 6) may be formed using other suitable conventions methods and mechanisms in alternative example embodiments. For instance, features of the modified substrate 14′ may be formed by embossing, perforating, or otherwise suitably processing the substrate 14.

The speed of the rotation of the drive roll 26 and the texturizing drum 36 may be set at various speeds with respect to each other. In some example embodiments, the speed of the rotation of the drive roll 26 and the texturizing drum 36 may be the same. In other example embodiments, the speed of the rotation of the drive roll 26 and the texturizing drum 36 may be different. For example, in some embodiments, the speed of the texturizing drum 36 may be less than the speed of the drive roll 26 to provide for overfeeding of the substrate 14 on the pattern surface 34 on the texturizing drum 36. Such overfeeding may be used to provide varied properties in the modified substrate 14′.

As shown in FIG. 3, in certain example embodiments, pattern surface 34 may be formed on texturizing drum 36, e.g., by stamping, machining, etc. It should be appreciated however that other mechanisms may be used to create the pattern surface 34. For example, a foraminous belt or wire (not shown) may be used which includes holes or three-dimensional features formed in the belt or wire at appropriate locations. Alternatively, flexible rubberized belts (not shown) which are impervious to the pressurized fluid entangling streams except in the location of holes may be used. Such belts and wires are well known to those of ordinary skill in the art as are the mechanisms for driving and controlling the speed of such belts and wires.

After the fluid entanglement occurs from the fluid entangling streams 44 by the fluid entanglement device 32, the modified substrate 14′ becomes a hydroentangled web. The method 10 may further include removing the modified substrate 14′ from the pattern surface 34 and drying the hydroentangled web to provide a three-dimensional nonwoven material. Drying of the material may occur through known techniques by one of ordinary skill in the art. In example embodiments where the precursory web includes binder fibers, the drying of the nonwoven material may activate the binder fibers. Activating the binder fibers may assist with the preservation of the three-dimensionality of the modified substrate 14′ by helping to preserve the geometry of features that extend away from the material.

As is the case with manufacturing various materials, the modified substrate 14′ may also be slit by a slitting device 15, as illustrated in FIG. 1. It can be common to manufacturing a material, such as a fluid entangled modified substrate 14′ described herein, to be at a greater width than will be used in various end use applications. Thus, the modified substrate 14′ may be slit by slitting techniques and apparatuses as known by those having ordinary skill in the art to provide multiple webs 12 (three such webs being shown in FIG. 1). The webs 12 may be slit in-line during the manufacturing of the modified substrate 14′ or may be slit in an off-line procedure, or later as part of another manufacturing asset seeking to utilize a certain width of the modified substrate 14′, and thus, one of the webs 12. The webs 12 may also be referred to as a zoned web 12, for purposes described further herein based on the features of the web 12.

Modified substrate 14′ may include zones with different morphological features. In various example embodiments, the morphological features may include one or more of: discrete perforated zones; discrete depression and/or protrusion zones, e.g., with depressions and/or hollow protrusions (such as those formed by embossing or other such web-modification processes); discrete apertures (whether integrally formed during web formation or formed through post web-formation processes); and/or discrete filled protrusions extending above, or below depending on orientation, a generally planar surface of the web. As a particular example, the modified substrate 14′ may include a first feature zone 70 with one or more first features and a second feature zone 72 with one or more second features. In the example embodiment shown in FIG. 1, the features in the first feature zone 70 may be apertures that extend through the modified substrate 14′, and the features in the second feature zone 72 may also be apertures that extend through the modified substrate 14′. The apertures in the first and second feature zones 70, 72 may assist with transferring fluids, such as body exudates, through the modified substrate 14′. However, while described in greater detail below in the context of apertures, it will be understood that each of first and second feature zones 70, 72 may include one or more alternative structural features in other example embodiments. For instance, each of first and second feature zones 70, 72 may include one or more of embossments, projections, depressions, perforations, protrusions, recesses, apertures, and the like. Thus, each of first and second feature zones 70, 72 may include discrete structural features on the modified substrate 14′, and the structural features in first feature zone 70 may be spaced apart from the structural features in second feature zone 72 on the modified substrate 14′. In certain example embodiments, the structural features in first feature zone 70 may be sized and/or formed differently than the structural features in second feature zone 72. Thus, e.g., the structural features in first feature zone 70 may provide different performance characteristics than the structural features in second feature zone 72. Moreover, each of first and second feature zones 70, 72 may be separately arranged, sized, shaped, and/or configured to provide a respective performance characteristic therein.

As discussed in greater detail below, the surface 34 may also include features for assisting with slitting of modified substrate 14′. Moreover, as described below, modified substrate 14′ may include discrete feature zones, e.g., first and second zones 70, 72, that are spaced apart on modified substrate 14′. For conciseness, the features of first and second zones 70, 72 will be referred to as apertures herein, but this description should not be construed to limit such features of first and second zones 70, 72 to only apertures. Due to the arrangement of first and second zones 70, 72 on modified substrate 14′, there may be gaps or spaces between the discrete aperture zones 70, 72 on modified substrate 14′ across the lateral direction 23.

In particular example embodiments, the location of the slitting between the discrete apertured zones 70, 72 can be critical for converting and performance of the resulting slitted substrate—for example, to ensure a sufficient width of regions or lanes of the slitted substrate on either side discrete apertured zones 70, 72. Utilizing a registration system to control the slitting process by identifying features of the apertured zones 70, 72 and adjusting a relative location of the slitting blades and the modified substrate 14′ can be a challenge at high speeds. For example, the features of the apertured zone may not be amenable for identification by a registration system at desired processing speeds, or such features of the apertured zone may have a variability that renders consistent identification difficult. Or, ensuring that such features of the apertured zones 70, 72 include features amenable for registration at high speeds can reduce the desired performance of the zones 70, 72 for their main functional purpose.

Accordingly, where such systems are utilized, tracking the relative position between the modified substrate 14′ and the knives used for slitting the modified substrate 14′ between the discrete aperture zones 70, 72 on modified substrate 14′ (i.e., at the gaps between the discrete aperture zones 70, 72 on modified substrate 14′) can be difficult, particularly at desired converting speeds. These systems can result in widths for the regions of lanes of the slitted substrate on either side of the discrete apertured zones 70, 72 being too small or too large.

Further, post processing the modified substrate 14′ to include registration features—such as applying colored or UV sensitive inks, bonds forming identifiable bonded regions, and the like—can also result in undesirable outcomes. For instance, inconsistent placement of the post processed registration features relative to the apertured zones can also result in poor alignment between the actual relative locations of the slitting blades and the modified substrate 14′ and a desired alignment between the slitting blades and the modified substrate 14′. Such conditions can also result in widths for the regions of lanes of the slitted substrate on either side of the discrete apertured zones being too small or too large.

Thus, as described in greater detail below, the pattern surface 34 may include one or more dedicated reference features—for example, a reference line 90 (FIG. 2) that may be used to form a plurality of dedicated reference features 96 (FIG. 6) on modified substrate 14′. Such dedicated reference features may be apertures or protrusions—whether discrete protrusions separate from each other or one continuously extending protrusion. For conciseness, features 96 will be referred to as apertures 96 herein, but this description should not be construed to limit such features 96 to only apertures. The dedicated reference apertures 96 may be used by the registration system to control the slitting process. Since the dedicated reference apertures 96 were formed at the time of material formation—and more specifically, at the time of formation of the zones 70, 72, the distance between the dedicated reference apertures 96 and the zones 70, 72 is fixed and there is no room for error of placement of a post-processed registration feature. Additionally, utilizing dedicated reference apertures 96 for registering desired cutting locations allows for the zones 70, 72 to be designed only based performance of the zones 70, 72 for desired functionality. In this way, the reference apertures 96 may thus advantageously provide for accurate registration of nonwoven webs with discrete aperture zones while allowing for superior performance of the material with respect to the function of zones 70, 72.

Although depicted in FIG. 6 as zones 70, 72 alternating in both the longitudinal direction 22 and the lateral direction 23, it should be understood that this is only one example configuration of a substrate 14′ whereby techniques of the present disclosure would be useful. Alternative configurations contemplated by the present disclosure include where the zones 70, 72 may be either continuous or discontinuous in the longitudinal direction 22 but do not alternate in the longitudinal direction 22. Rather, the zones 70, 72 may be maintained within discrete lanes separated across the width of the modified substrate 14′ in the lateral direction 23. In such configurations, space may be provided between laterally adjacent zones 70, 72. In still further contemplated example embodiments, the modified substrate 14′ may include only a single type of the zones 70, 72 (whether continuous or discontinuous in the longitudinal direction 22), with such single zone types being provided in spaced apart lanes as shown in FIG. 6.

Turning back the discussion to an example methodology for modifying the substrate 14 in a fluid entanglement process, attention is drawn to FIG. 2. FIG. 2 illustrates a top plan view of a portion of the pattern surface 34, in which the pattern surface 34 is depicted in a laid flat orientation for purposes of clarity. The pattern surface 34 can include a plurality of pattern lanes 48, 48′, 48″. The pattern surface 34 includes at least two pattern lanes 48, 48′. For example, the example embodiment illustrated in FIG. 2 includes three pattern lanes 48, 48′, 48″. The pattern lanes 48, 48′, 48″ may include at least two pattern zones 50, 52. For example, each pattern lane 48, 48′, 48″ may each include a first pattern zone 50 and a second pattern zone 52. As shown in FIG. 2, the pattern lanes 48, 48′, 48″ may be configured such that the first pattern zone 50 and second pattern zone 52 are adjacent to one another in the machine direction 22 and repeating in an alternating fashion, such that each first pattern zone 50 is adjacent two second pattern zones 52 in the machine direction 22, and such that each second pattern zone 52 is adjacent two first pattern zones 50 in the machine direction 22. It is contemplated, and within the scope of example aspects of the disclosure that one or more pattern lanes 48, 48′, 48″ may include more than two different pattern zones 50, 52.

The first pattern zone 50 may include different characteristics than the second pattern zone 52. The different characteristics between the first pattern zone 50 and the second pattern zone 52 may provide for different substrate characteristics in the modified substrate 14′ that is created from the pattern surface 34 and that relate to handling characteristics of the modified substrate 14′. As an example, the first pattern zone 50 may produce a first zone 70 (labeled in FIG. 6) of the modified substrate 14′ that has an open area that is greater than an open area of the second zone 72 (labeled in FIG. 6) of the modified substrate 14′ produced by the second pattern zone 52. For purposes herein, “open area” refers to an amount of open area as measured by the analysis techniques in the Material Sample Analysis Test Method as described in the Test Methods section herein. Other substrate characteristics that may differ between the first zone 70 and the second zone 72 of the modified substrate 14′ that are produced by the first pattern zone 50 and the second pattern zone 52, respectively, may provide different web handling characteristics of the modified substrate 14′ and may include, for example, differences in basis weight, density, tensile strength, bulk thickness, surface texture, and/or urine wicking properties, among other characteristics.

FIG. 4 provides a detailed portion of the first pattern zone 50 of the pattern surface 34 from the first pattern lane 48 from the example pattern surface 34 of FIG. 2. The portion of the first pattern zone 50 in FIG. 4 is labeled “4” in FIG. 2. In an example embodiment, the first pattern zone 50 of the pattern surface 34 may include a plurality of forming holes 54, a plurality of projections 56, and a plurality of connecting ligament forming areas 69. The connecting ligament forming areas 69 may be disposed between the plurality of forming holes 54 and the plurality of projections 56 and may generally be areas of the pattern surface 34 that are neither a forming hole 54 nor a projection 56.

As will be discussed in more detail below, the geometry, spacing, and orientation of the forming holes 54, the projections 56, and the connecting ligament forming areas 69 may correspond to the formation of the nodes 74, apertures 76, and connecting ligaments 78 in the modified substrate 14′. The alignment and orientation of these forming holes 54, projections 56, and connecting ligament areas 69 may provide for beneficial properties, such as tensile strength and desired necking for processability of the materials, while still allowing for a highly-open material and thus achieving beneficial fluid-handling properties. Although such alignments and orientations are described with respect to the specific pattern of the first pattern zone 50 of the pattern surface 34 in FIG. 4, it should be understood that other patterns for the first pattern zone 50 for pattern surfaces are contemplated by example aspects of the present disclosure and may achieve such described alignments and orientations in other patterns.

As depicted in FIG. 4, the first pattern zone 50 of the pattern surface 34 may include a plurality of forming holes 54 that correspond to the shape and pattern of the nodes 74 of the modified substrate 14′. While the forming holes 54 depicted in FIG. 4 are round, it should be understood that any number of shapes and combination of shapes may be used depending on the end use application. Examples of additional or alternative possible forming hole 54 shapes include, but are not limited to, ovals, crosses, squares, rectangles, diamond shapes, hexagons and other polygons.

The forming holes 54 may be arranged in a plurality of columns 55 (three columns 55 labeled in FIG. 4) that extend in the longitudinal direction 57 of the pattern surface 34. The longitudinal direction 57 of the pattern surface 34 may correspond to a circumferential direction, for example, if the pattern surface 34 is part of a cylindrical texturizing drum 36. The columns 55 of forming holes 54 may be formed of longitudinally adjacent forming holes 54. Forming holes 54 are longitudinally adjacent if a line 63 drawn between centers of forming holes 54 does not pass through any projections 56 or any other forming holes 54 and forms an angle with respect to the longitudinal direction 57 of less than forty-five degrees (45°), such as less than about twenty degrees (20°), such as less than about ten degrees (10°), such as less than about five degrees (5°). No angle is shown in FIG. 4 because the angle formed by line 63 with respect to the longitudinal direction 57 is zero degrees (0°). Similarly, the forming holes 54 may also be arranged in columns (also referred to as “rows” in such arrangements) that extend in the lateral direction 61 of the pattern surface 34 if a line drawn between centers of forming holes 54 does not pass through any projections 56 or any other forming holes 54 and forms an angle with respect to the lateral direction 61 of the pattern surface 34 of less than forty-five degrees (45°), such as less than about twenty degrees (20°), such as less than about ten degrees (10°), such as less than about five degrees (5°). The orientation of the forming holes 54 in the pattern surface 34 determines the orientation of the nodes 74 in the modified substrate 14′, as fibers from the substrate 14 are pushed into the forming holes 54 by the fluid jets 44 during fluid entanglement discussed above.

In some example embodiments, it may be preferable for the first pattern zone 50 of the pattern surface 34 to have at least three (3) columns 55 of forming holes 54, at least four (4) columns 55, at least five (5) columns 55, at least six (6) columns 55, or at least seven (7) or eight (8) columns 55, which extend substantially in the longitudinal direction 57. The columns 55 of forming holes 54 that extend substantially in the longitudinal direction 57 of the pattern surface 34 may have a length that spans the entire machine directional length of the first pattern zone 50 of the pattern surface 34 or may form only a portion of the pattern surface 34 length in the longitudinal direction 57 (such as a portion of the circumference of the pattern surface 34).

The pattern surface 34 may also include a plurality of projections 56 extending away from the base plane 42 of the pattern surface 34. As depicted in FIG. 4, the projections 56 may be configured in a pyramidal geometry, however, the projections 56 may be in various other geometries, cross-sectional shapes, spacings, and orientations. In some example embodiments, the plurality of projections 56 may decrease in cross-sectional area as the projections 56 extend further away from the base plane 42 of the pattern surface 34. For example, the pyramidal shape of the projections 56 depicted in FIG. 4 decrease in area the further the projection 56 extends away from the base plane 42 of the pattern surface 34.

The projections 56 in the first pattern zone 50 of the pattern surface 34 may form the apertures 76 in the first zone 70 of the modified substrate 14′ (labeled in FIG. 6), as fibers from the substrate 14 are pushed around the projections 56 in the first pattern zone 50 by the fluid jets 44 during fluid entanglement discussed above. Thus, the orientation, density, and size of the projections 56 in the first pattern zone 50 of the pattern surface 34 may correspond to the orientation, density, and size of the apertures 76 in the first zone 70 of the modified substrate 14′.

The projections 56 may be arranged in a plurality of columns 59 (three columns 59 labeled in FIG. 4) that extend in the longitudinal direction 57 of the pattern surface 34. The columns 59 of projections 56 may be formed of a series of connected, longitudinally adjacent projections 56. Projections 56 are longitudinally adjacent where a line (such as line 65a or 65b in FIG. 4) does not pass through any forming holes 54 or any other projections 56 and spans across only a single connecting ligament forming area 69 and forms an angle with respect to the longitudinal direction 57 of the pattern surface 34 of less than about forty-five degrees (45°). The centers of the projections 56 may be the geometric centers of the projections 56. Similarly, the projections 56 may also be laterally adjacent when if a line drawn between centers of projections 56 does not pass through any forming holes 54 or any other projections 56 and the line only spans across a single connecting ligament forming area 69 and forms an angle with respect to the lateral direction 61 of the pattern surface 34 of less than forty-five degrees (45°).

In some example embodiments, on the lateral sides of the first pattern zone 50, the pattern surface 34 may include one or more areas 60a, 60b that are substantially free from projections 56. The areas 60a, 60b may correspond to the side zones 80a, 80b in the modified substrate 14′ (two side zones 80a, 80b labeled in FIG. 6). In some example embodiments, the areas 60a, 60b corresponding to the side zones 80a, 80b may include apertures 71. However, in certain example embodiments, if included, the apertures 71 in the areas 60a, 60b may be smaller in cross-sectional area than the forming holes 54 in the first pattern zone 50 of the pattern surface 34 and may assist with fluid removal during the fluid entangling process. For example, an average area of the apertures 71 in the areas 60a, 60b may be less than an average area of the forming holes 54 in the first pattern zone 50. In some example embodiments, an average area of the apertures 71 can be between about one millimeter squared (1.0 mm²) to about one and a half millimeters squared (1.5 mm²). Depending on the density of the apertures 71 in the areas 60a, 60b and the area of the apertures 71, in some example embodiments, the apertures 71 may provide from about fifteen percent (15%) to about fifty percent (50%) of the area of the areas 60a, 60b. The apertures 71 in the areas 60a, 60b may lead to formation of micro-bumps in the modified substrate 14′. The area of the base plane 42 of the forming surface 50 between the apertures 71 in zones 60a, 60b may form micro-apertures and/or areas of lower fiber density in the modified substrate 14′.

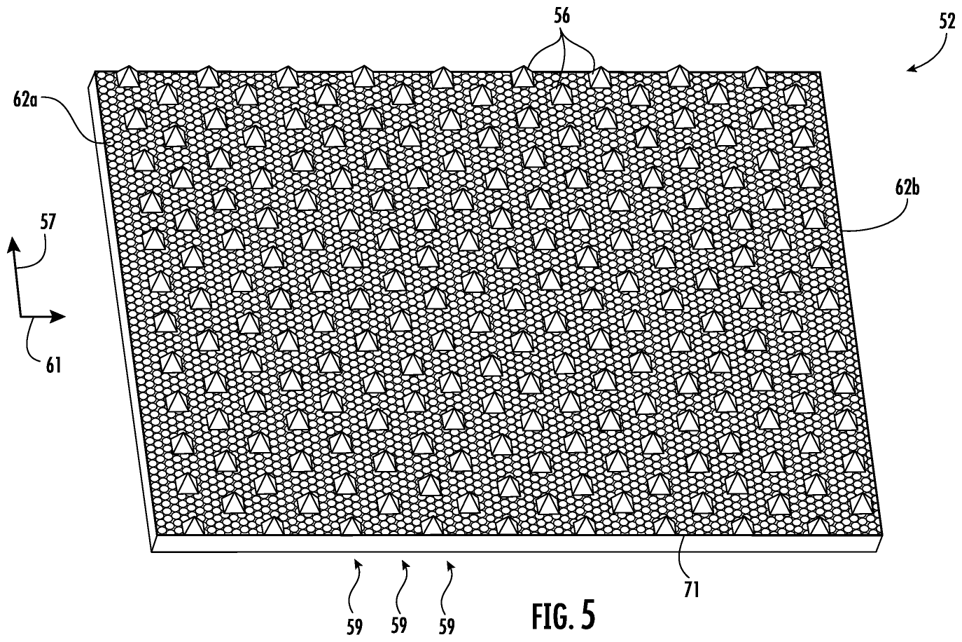

Turning now to FIG. 5, a portion of the second pattern zone 52 of the pattern surface 34 of FIG. 2 is illustrated. The portion of the second pattern zone 52 in FIG. 5 is labeled “5” in FIG. 2. The second pattern zone 52 may include projections 56 that extend away from the base plane 42 of the pattern surface 34. The projections 56 in the second pattern zone 52 may be the same size and/or height as the projections 56 in the first pattern zone 50. In some example embodiments, the projections 56 in the second pattern zone 52 may be a different size and/or height as the projections 56 in the first pattern zone 50.

The projections 56 can be arranged in a plurality of columns 59 (three columns 59 labeled in FIG. 5) that extend in the longitudinal direction 57 of the pattern surface 34. The columns 59 of projections 56 may can be formed of a series of connected, longitudinally adjacent projections 56, as described above with respect to the projections 56 in the first pattern zone 50 of the pattern surface 34. In some example embodiments, the first pattern zones 50 may include six, seven, or eight or more columns 59 of projections 56 extending in the longitudinal direction 57. In some example embodiments, a majority of the plurality of columns 59 of projections 56 extending in the longitudinal direction 57 are laterally offset from a nearest adjacent column 55 of forming holes 54 extending substantially in the longitudinal direction 57.

The projections 56 in the second pattern zone 52 of the pattern surface 34 may form the apertures 76 in the second zone 72 of the modified substrate 14′ (labeled in FIG. 6), as fibers from the substrate 14 are pushed around the projections 56 in the second pattern zone 52 by the fluid jets 44 during fluid entanglement discussed above. Thus, the orientation, density, and size of the projections 56 in the second pattern zone 52 of the pattern surface 34 may correspond to the orientation, density, and size of the apertures 76 in the second zone 72 of the modified substrate 14′.

In some example embodiments, on the lateral sides the second pattern zone 52, the pattern surface 34 may include one or more areas 62a, 62b that are substantially free from projections 56. The areas 62a, 62b may correspond to the side zones 82a, 82b in the modified substrate 14′ (two side zones 82a, 82b labeled in FIG. 6). In some example embodiments, the areas 62a, 62b corresponding to the side zones 80a, 80b may include apertures 71. However, in example embodiments, if included, the apertures 71 in the areas 62a, 62b may be smaller in cross-sectional area than the forming holes 54 in the first pattern zone 50 of the pattern surface 34 and may assist with fluid removal during the fluid entangling process. For example, an average area of the apertures 71 in the areas 62a, 62b may be less than an average area of the forming holes 54 in the first pattern zone 50. The apertures 71 in the areas 62a, 62b may lead to formation of micro-bumps in the modified substrate 14′.

The second pattern zone 52 of the pattern surface 34 can also include a plurality of apertures 71 disposed between projections 56 in the second pattern zone 52. The apertures 71 in the second pattern zone 52 may be similar to the apertures 71 described above with respect to areas 62a, 62b on the lateral sides of the second pattern zone 52. The apertures 71 may assist with fluid management during the fluid entanglement process. In some example embodiments, the second pattern zone 52 can be configured to not include any apertures 71.

As illustrated in FIG. 2, in some example embodiments, the pattern surface 34 may also include one or more transition pattern zones 51. A transition pattern zone 51 in the pattern surface 34 may be disposed between the first pattern zone 50 and the second pattern zone 52 in the longitudinal direction 57 of the pattern surface 34. The transition pattern zone 51 may be an area of the pattern surface 34 that is free from projections 56 or at least has less projections 56 than the first pattern zone 50. In some example embodiments, the transition pattern zone 51 may include apertures similar to the apertures 71 described above with respect to areas 60a, 60b on the lateral sides of the first pattern zone 50. The transition pattern zone 51 of the pattern surface 34 may correspond to the transition zone 73 (labeled in FIG. 6) of the modified substrate 14′ after the substrate 14 is modified by the fluid entanglement apparatus 32. Although not depicted, in some example embodiments, the transition pattern zone 51 may also include forming holes 54 such as described above with respect to the first pattern zone 50. Thus, e.g., the first and second pattern zones 50, 52 may be discrete or separate from one another on pattern surface 34.

In an example embodiment, the pattern lanes 48, 48′, 48″ can be configured such that the first pattern zone 50 in the first pattern lane 48 is substantially the same as the first pattern zone 50 in the second pattern lane 48′. In some example embodiments, the first pattern zone 50 in the third pattern lane 48″ may be substantially the same as the first pattern zone 50 in the second pattern lane 48′ and/or substantially the same as the first pattern zone 50 in the first pattern lane 48. Similarly, in some example embodiments, the pattern lanes 48, 48′, 48″ may be configured such that the second pattern zone 52 in the first pattern lane 48 is substantially the same as the second pattern zone 52 in the second pattern lane 48′. In some example embodiments, the second pattern zone 52 in the third pattern lane 48″ may be substantially the same as the second pattern zone 52 in the second pattern lane 48′ and/or substantially the same as the second pattern zone 52 in the first pattern lane 48.

Alternatively, it is contemplated that the first pattern zone 50 in the first pattern lane 48 may be configured differently than the first pattern zone 50 in a second pattern lane 48′ and/or the first pattern zone 50 in the third pattern lane 48″. It is also contemplated that the second pattern zone 52 in the first pattern lane 48 may be configured to be different from the second pattern zone 52 in the second pattern lane 48′ and/or the second pattern zone 52 in the third pattern lane 48″.

As noted above, and referring back to FIG. 2, the pattern surface 34 may also include a reference line 90. Reference line 90 may include a plurality of reference features 92. As noted above, such dedicated reference features in the modified substrate 14′ may be apertures or protrusions—whether discrete apertures or protrusions separate from each other or one continuously extending aperture or protrusion. Features 92 on the pattern surface 34 that may form the corresponding apertures or protrusions in the modified substrate 14′ may be projections protruding from the forming surface 34, apertures extending through the forming surface 34, or depressions within the forming surface 34 but which do not extend completely through the forming surface 34. Projections on the forming surface 34 may form corresponding apertures in the modified substrate 14′. Apertures in the forming surface 34 may form corresponding projections in the modified substrate 14′. Depressions in the forming surface 34 may form corresponding projecting, densified regions on the modified substrate 14′. Each of these apertures, projections, and projecting, densified regions may be detectable by a registration system.

For conciseness, features 92 will be referred to as projections 92 herein, but this description should not be construed to limit such features 92 to only projections as indicated above. The projections 92 in the reference line 90 of the pattern surface 34 may form a plurality of apertures 96 in a reference line index 94 of the modified substrate 14′ (labeled in FIG. 6), as fibers from the substrate 14 are pushed around the projections 92 in the reference line 90 by the fluid jets 44 during fluid entanglement discussed above. Thus, the orientation, density, and size of the projections 92 in the reference line 90 of the pattern surface 34 may correspond to the orientation, density, and size of the apertures 96 in the reference line index 94 of the modified substrate 14′.

In example embodiments, the projections 92 of the reference line 90 may be spaced from the one of pattern lanes 48, 48′, 48″, such as first pattern lane 48, e.g., along a lateral direction perpendicular to the longitudinal direction 57, by no less than twenty millimeters (20 mm) and no greater than one hundred millimeters (100 mm). Moreover, the projections 92 of the reference line 90 may be uniformly spaced along the longitudinal direction 57 on the pattern surface 34. For instance, each projection 92 may be spaced from adjacent projections 92 within the reference line 90 along the longitudinal direction 57 by no less than a half millimeter (0.5 mm) and no greater than ten millimeters (10 mm). Thus, e.g., the reference line 90 may be continuous along the longitudinal direction 57 of pattern surface 34. Moreover, respective projections 92 of the reference line 90 may be positioned adjacent each of first pattern zone 50, second pattern zone 52, and transition pattern zone 51 on the pattern surface 34 along the lateral direction such that reference line 90 is continuous across first pattern zone 50, second pattern zone 52, and transition pattern zone 51 along the longitudinal direction 57 for each of the pattern lanes 48, 48′, 48″. In example embodiments, the projections 92 of the reference line 90 may be pins having any suitable cross-section, including circular, ovular, triangular, square, and more. A diameter or smallest dimension of the projections 90 may be as small as one-tenth of a millimeter (0.1 mm) and no greater than ten millimeters (10 mm).

The projections 92 of the reference line 90 may be positioned at a side portion or edge of the pattern surface 34. In contemplated example embodiments according to the present disclosure, the reference line 90 is provided at a single side portion or edge of the pattern surface 34. Moreover, as shown in FIG. 2, the pattern surface 34 may include two reference lines 90 in certain example embodiments. Each of the reference lines 90 may be positioned at a respective side portion or edge of pattern surface 34. Such arrangement may advantageously form apertures 96 in the reference line 15 index 94 at edges of the modified substrate 14′. However, it will be understood that the placement of reference line(s) 90 in FIG. 2 is provided by way of example only and that other positions, such as at a central portion of pattern surface 34, are within the scope of the present disclosure.

Wherever placed on the pattern surface 24, in some example embodiments, the reference line 90 may preferably be separated and discrete from the pattern zones 50, 51, and 52. Such pattern zones 50, 51, and/or 52 may provide desired functional performance characteristics to the modified substrate 14′. By providing discrete reference line(s) 90, a discrete registration feature may be formed in the modified substrate 14′ without imposing requirements on the pattern zones 50, 51, and 52 in order to be useful for registration which could affect the performance characteristics of the modified substrate 14′. Additionally, by utilizing such discrete reference line(s) 90 on the pattern surface 34, a discrete reference feature 94 is integrally formed in the modified substrate 14′ in the same manner or at the same time as the zones 70 and/or 72 by pattern zones 50, 51, and 52. Having such a discrete reference feature 94 formed in the modified substrate 14′ provides an advantage of ensuring consistent dimensional relation between the discrete reference feature 94 and other portions of the modified substrate 14′—for example zones 70, 72. In contrast, applying reference features post-formation of substrate 14′ requires additional equipment to ensure consistent placement of such features on the modified substrate 14′ and can be challenging to achieve such a result even with such additional equipment.

Providing a discrete reference feature 94 formed in the modified substrate 14′ is particularly advantageous where the modified substrate 14′ is laminate material, such as that produced by hydroentangling the substrate 14 with a support web (as described in U.S. Pat. No. 9,327,473 to Scott Kirby et al.). In such example embodiments, these laminates are prone to distortion when placed under tension (e.g., necking, curling, etc.) creating challenges for handling such material in a consistent manner in commercial converting equipment. By the providing a discrete reference feature 94 formed in the modified substrate 14′, the reference feature is similarly subject the same forces and is better able to provide a consistent reference point for registration than other, alternative reference feature options.

The projections 92 of the reference line 90 may be arranged in a column that extends in the longitudinal direction 57 of the pattern surface 34. When the pattern surface 34 is cylindrical, as shown in FIG. 4, the projections 92 of the reference line 90 may be arranged in ring that extends circumferentially around of the cylindrical texturizing drum 36. Moreover, first and second pattern zones 50, 52 may be circumferentially spaced on around of the cylindrical texturizing drum 36, with reference line 90 axially spaced from adjacent zones 50, 52 on the cylindrical texturizing drum 36. In example embodiments, the column of projections 92 in the reference line 90 may be formed of longitudinally adjacent projections 92 that are distributed, e.g., in a rectilinear pattern, on pattern surface 34. Because the orientation, density, and size of the projections 92 in the reference line 90 of the pattern surface 34 determines the orientation, density, and size of the apertures 96 in the reference line index 94 of the modified substrate 14′, the apertures 96 in the reference line index 94 of the modified substrate 14′ may also be arranged in a column that extends in the lateral direction 23 of the modified substrate 14′. Thus, e.g., as shown in FIG. 6, the column of apertures 96 in the reference line index 94 may be formed of longitudinally adjacent apertures 96 that are distributed, e.g., in a rectilinear pattern, on modified substrate 14′ in the lateral direction 23. As one illustrative example, the column of apertures 96 may be formed of two, or three, or more laterally adjacent columns of apertures.

In example embodiments, apertures 96 in the reference line index 94 may be uniformly distributed along the machine direction 22 of the modified substrate 14′. Thus, e.g., the reference line index 94 may be continuous along the longitudinal direction 22 of the modified substrate 14′. Moreover, respective apertures 96 of reference line index 94 may be positioned adjacent each of first zone 70, second zone 72, and transition zone 73 on modified substrate 14′ along the lateral direction 23 such that reference line index 94 is continuous across first zone 70, second zone 72, and transition zone 73 along the longitudinal direction 22. In example embodiments, adjacent apertures 96 of reference line index 94 may be spaced apart in longitudinal direction 22 by no less than a tenth of a millimeter (0.1 mm) and no greater than one and a half millimeters (1.5 mm), such as about a half millimeter (0.5 mm).

In contrast to reference line index 94, first and second zones 70, 72 may be discrete and spaced from each other on modified substrate 14′ with transition zones 73 therebetween. The arrangement of apertures 96 in the reference line index 94 may facilitate registration of the modified substrate 14′, e.g., during a slitting process in the manner described above.

Turning back to FIG. 1, as noted above, the method 10 may include supplying or feeding the continuous modified substrate 14′ to slitting device 15. From pattern surface 34, modified substrate 14′ may include the series of apertured regions, namely first and second zones 70, 72, as well as a reference line index 94. Method 10 may also include features for assisting with registering reference line index 94, e.g., to assist with accurate slitting of the continuous modified substrate 14′ at slitting device 15. For instance, based upon detection of reference line index 94, the method 10 may affect a change in position of the modified substrate 14′, e.g., along a cross-machine direction perpendicular to the machine direction 22, with respect to slitting device 15 and thereby adjust a location on the modified substrate 14′ where the modified substrate 14′ is slit by slitting device 15.

Utilizing slitting device 15 with registration of reference line index 94 may advantageously allow slitting device 15 to slit the modified substrate 14′ between adjacent lanes of aperture zones on modified substrate 14′, such as first and second zones 70, 72. For example, the slitting device 15 may slit the modified substrate 14′ into three (3) or more zoned webs by slitting the modified substrate 14′ between adjacent lanes 88, 88′, 88″ of aperture zones. Thus, e.g., each zoned web 12 from modified substrate 14′ after splitting device 15 may include first and second zones 70, 72 as discussed above.

To adjust the position of the modified substrate 14′, e.g., along the cross-machine direction perpendicular to the machine direction 22, with respect to slitting device 15, the method 10 may, e.g., adjust a tension of the modified substrate 14′. For instance, the method 100 may further include rolls that assist in feeding the modified substrate 14′ in the machine direction 22, and a rotational speed of the rolls may be adjusted to modify the tension of the modified substrate 14′. As another example, the rolls may be translated, e.g., along the cross-machine direction, to adjust the position of the modified substrate 14′ along the cross-machine direction. As another example, knives within slitting device 15 may be translated along the cross-machine direction to adjust the cutting by slitting device 15. Other known techniques, apparatuses, and mechanisms for adjusting the position of the modified substrate 14′, e.g., along the cross-machine direction perpendicular to the machine direction 22, relative to slitting device 15 are also within the scope of the present subject matter. In general, it should be understood that the particular mechanism or method in which a change in position of the modified substrate 14′ and/or the slitting device 15 to adjust the slitting of the modified substrate 14′ by the slitting device 15 is not critical as long as such the function is included.

Referring back to FIG. 1, according to example aspects of the present disclosure, the method 10 may further include a registration system 110 that may be used to carry out a registration process for registering the modified substrate 14′. The registration system 110 may include a registration processing device 120 which itself may include one or more processors 122, one or more data storage devices 124, and at least one communication device 126. The registration system 110 may further include externally connected devices, such as image capture devices 130, 132 and the system 110 may further be connected at least to feed rolls, slitting device 15, or other operative components of method 10 for adjusting the slitting of the modified substrate 14′ by the slitting device 15. The one or more processors 122 may be configured to implement functionality and/or process instructions for execution within device 120. The one or more processors 122 may be capable of processing instructions stored in data storage device(s) 124. Examples of processor(s) 122 may include, any one or more of a microprocessor, a controller, a digital signal processor (DSP), an application specific integrated circuit (ASIC), a field-programmable gate array (FPGA), or equivalent discrete or integrated logic circuitry.

The communication device 126 may effect communication between the components and modules of the device 120, as well as any external connected devices such as image capture devices 130, 132 and/or feed rolls 116. Each of components 122, 124, and 126 may be interconnected (physically, communicatively, and/or operatively) for inter-component communications. In some examples, communication channels (not shown) may extend between the components 122, 124, and 126, which may include a system bus, a network connection, an inter-process communication data structure, or any other method for communicating data.

Where communication device(s) 126 is included, device 120 may utilize communication device(s) 126 to communicate with external devices via one or more networks, such as one or more wired and/or wireless networks. Communication device(s) 126 may be a network interface card, such as an Ethernet card, an optical transceiver, a radio frequency transceiver, or any other type of device that can send and receive information. Other examples of such network interfaces may include Bluetooth®, 3G and WiFi radios computing devices as well as Universal Serial Bus (USB).

The one or more storage device(s) 124 may be configured to store information within device 120 during operation. Storage device(s) 124, in some examples, is described as a computer-readable storage medium. Storage device(s) 124 may be a temporary memory, meaning that a primary purpose of storage device(s) 124 is not long-term storage. Storage device(s) 124, in some examples, may be a volatile memory, for example random access memories (RAM), dynamic random access memories (DRAM), static random access memories (SRAM), and other forms of volatile memories known in the art. In some examples, storage device(s) 124 may be used to store program instructions for execution by processor(s) 122. Storage device(s) 124, in one example, may be used by software or applications running on device 120 (e.g., modules 121, 123, and 125) to temporarily store information during program execution.

Storage device(s) 124, in some examples, also include one or more computer-readable storage media. Storage device(s) 124 may be configured to store larger amounts of information than volatile memory. Storage device(s) 124 may further be configured for long-term storage of information. In some examples, storage device(s) 124 include non-volatile storage elements such as magnetic hard discs, optical discs, floppy discs, flash memories, or forms of electrically programmable memories (EPROM) or electrically erasable and programmable (EEPROM) memories.

The one or more data storage device(s) 124 may include one or more modules stored therein which may help to perform the registration function of the system 110, for example an image capture module 121, and image modification module 123, and a registration module 125. Although the modules 121, 123, and 125 are described as carrying out a number of functions of the system 110, the functions attributed to such modules may be implemented in more, fewer, and/or different modules. Additionally, in some examples, the functions may not be distributed between physical or logical modules, but, instead, may be executed by, e.g., processors 122 based on instructions and data stored on storage device(s) 124. Further, although modules 121, 123, and 125 are illustrated as part of storage device(s) 124 in the example of FIG. 1, in other examples, the modules 121, 123, and 125 may be implemented separate from storage device(s) 124 including, e.g., implemented in discrete hardware components configured to carry out the functions attributed to the modules in the examples disclosed herein.

Image capture devices 130, 132 may be configured to capture digital images and communicate such captured images to device 120 for storage in the storage device(s) 124. Image capture devices 130, 132 may be digital devices including at least an optical system including a lens arrangement and an image sensor, such as charge-coupled device (CCD) sensor or a complementary metal-oxide-semiconductor (CMOS) sensor or the like. The image capture devices 130, 132 may further include a shutter and other components typical of image capture devices. Although not shown, the image capture devices 130, 132 may additionally include illumination devices for illuminating the location or object of which the image capture devices 130, 132 are configured to capture images. In at least some of these example embodiments, such illumination devices may be positioned opposite the image capture devices 130, 132, such that the illumination provides a back-lit illumination. For example, such illumination devices may be disposed on an opposite side of the modified substrate 14′ with respect to the image capture devices 130, 132 of FIG. 1.

The image capture module 121 may be configured to trigger image capture devices 130, 132 and obtain discrete images of the modified substrate 14′ (from image capture device 130) and the zoned webs 12 (from image capture device 132). The images captured from devices 130, 132 may be stored in the storage device(s) 124. The image capture module 121 may be generally configured to capture images of the modified substrate 14′ and zoned webs 12 at regular intervals, such as on a once per product basis, according to methods known in the art. For example, the image capture module 121 may be connected to feed rolls and may adjust the rate at which the image capture module 121 triggers the image capture devices 130, 132 as the speed of the modified substrate 14′ is increased or decreased. Additionally, initial setup of the trigger may be performed according to methods known in the art to ensure that the captured images correspond to the portions of the modified substrate 14′ which are ultimately disposed on a single product. For example, such captured images may be displayed on an output devices, and a manual adjustment of the trigger may be made to ensure proper timing of the image capture trigger. Alternatively, one or more preset parameters may be loaded into image capture module 121 and one or more image processing techniques may be used to determine a feature(s) of the captured images. Comparison of the one or more preset parameters and determined feature(s) of the captured images may be performed and an automatic adjustment of the trigger may be performed by image capture module 121 based on the comparison to ensure proper timing of the image capture trigger.

With the captured images stored in the storage device(s) 124, the image modification module 123 may perform one or more image processing modifications to the stored images. For example, the image modification module 123 may be configured to filter the captured images according to one or more filter parameters. Such a filtering process may help to minimize features of non-interest and highlight features of interest—for example, the reference line index 94 which may be continuous along the length of modified substrate 14′. The image modification module 123 may be further configured to accentuate the features of interest by performing one or more morphological process techniques on the captured images and/or the filtered captured images.

Finally, the registration module 125 may be configured to identify features of the modified captured images and to adjust the method 10 to ensure a proper registration of the modified substrate 14′ relative to the slitting device 15 is achieved. For example, the registration module 125 may be configured to identify or determine the reference line index 94 on the modified substrate 14′. According to some example embodiments, the registration module 125 may be configured to identify the apertures 96 within the reference line index 94 within the modified captured images—for example, along the length of the modified substrate 14′. Next, the registration module 125 may be configured to determine one or more metrics related to the determined features—such as a difference in locations—and, based on the one or more determined metrics, adjust the method 10 to ensure a proper registration of the modified substrate 14′ is achieved relative to the slitting device 15. Because reference line index 94 is continuous (in contrast the discrete, separate first and second zones 70, 72 on modified substrate 14′, registration module 125 may utilize continuous light transmittance through apertures 96 of the reference line index 94 to set and control operation of slitting device 15 to provide accurate and precise cutting of modified substrate 14′ with slitting device 15 into zoned webs 12.

Three-Dimensional Web with Nodes:

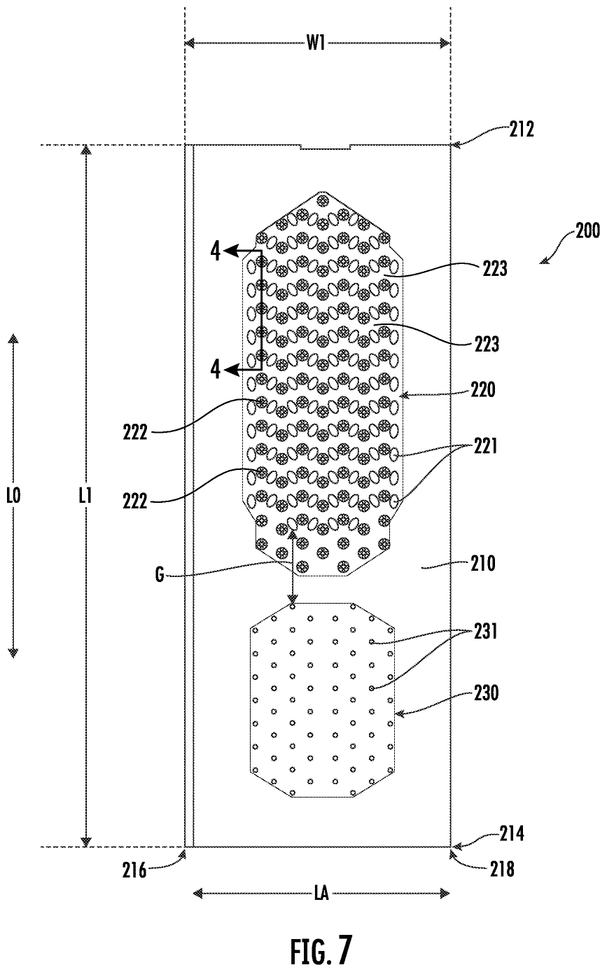

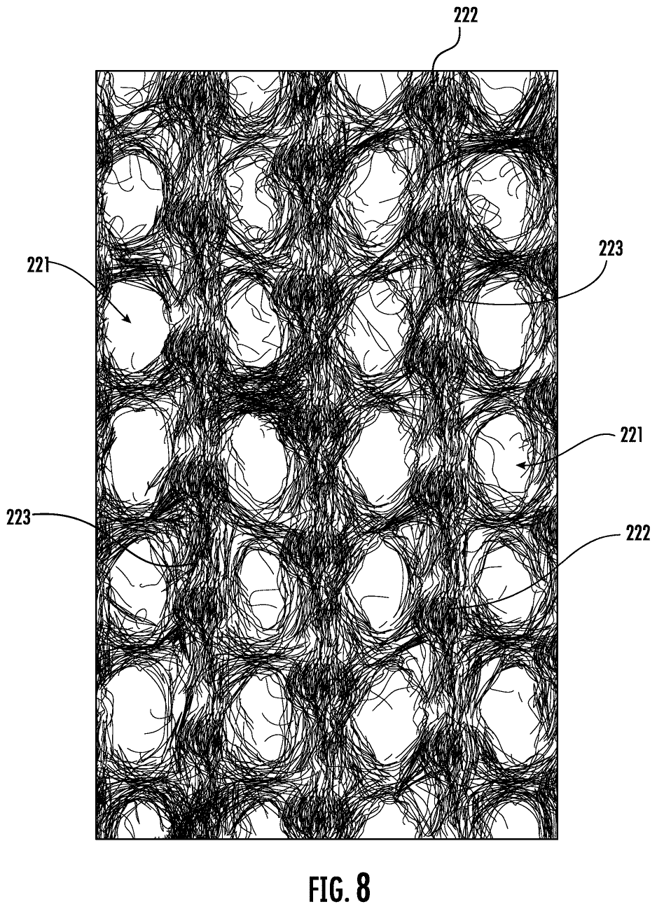

FIG. 7 is a top, plan view of a nonwoven material 200 according to an example embodiment of the present disclosure. In FIG. 7, nonwoven material 200 is shown in a flat or planar configuration. FIG. 8 is an image of a first feature zone 220 of nonwoven material 200. Nonwoven material 200 may be used in an absorbent article, e.g., as a bodyside liner. However, it will be understood that nonwoven material 200 may be used in any other article or garment in alternative example embodiments. For instance, nonwoven material 200 may be used in diapers, diaper pants, training pants, youth pants, swim pants, feminine hygiene products, including, but not limited to, menstrual pads or pants, incontinence products, medical garments, surgical pads and bandages, other personal care or health care garments, e.g., as an inner liner facing a wearer of such products, and wipes. As discussed in greater detail below, nonwoven material 200 may advantageously include discrete apertures zones, e.g., positioned centrally along a length of the nonwoven material 200.

As shown in FIG. 7, nonwoven material 200 includes a nonwoven fibrous web 210, e.g., formed with a plurality of fibers. Fibrous web 210 may define a lateral direction LA and a longitudinal direction LO. The lateral and longitudinal directions LA, LO may be perpendicular to each other. In certain example embodiments, longitudinal direction LO may correspond to the longitudinal direction 30, and lateral direction LA may correspond to the lateral direction 23. Fibrous web 210 may extend between a rear or first end portion 212 and a front or second end portion 214, e.g., along the longitudinal direction LO. Thus, first and second end portions 212, 214 may be spaced apart along the longitudinal direction LO. First end portion 212 of fibrous web 210 may be positioned at or adjacent a rear waist region of an absorbent article, and second end portion 214 of fibrous web 210 may be positioned at or adjacent a front waist region of an absorbent article. Fibrous web 210 may also extend between a first side portion 216 and a second side portion 218, e.g., along the lateral direction LA. Thus, first and second side portions 216, 218 may be spaced apart along the lateral direction LA.

A length L1 of fibrous web 210 may be defined between first and second end portions 212, 214 of fibrous web 210, and a width W1 of fibrous web 210 may be defined between first and second side portions 216, 218 of fibrous web 210. In certain example embodiments, the length L1 of fibrous web 210 may be greater than the width W1 of fibrous web 210. For example, the length L1 of fibrous web 210 may be no less than two times (2X) greater than the width W1 of fibrous web 210, such as no less than three times (3X) greater than the width W1 of fibrous web 210. Thus, fibrous web 210 may be elongated along longitudinal direction LO between the first and second end portions 212, 214.

Nonwoven fibrous web 210 may include at least one feature zone. For instance, as shown in FIG. 7, nonwoven fibrous web 210 may include a first feature zone 220 and a second feature zone 230. First feature zone 220 may be spaced apart from second feature zone 230, e.g., along the longitudinal direction LO. For example, first and second feature zones 220, 230 may be spaced apart by a gap G along the longitudinal direction LO. In certain example embodiments, the gap G may be no less than six millimeters (6 mm) and no greater than one hundred and thirty millimeters (130 mm), such as no less than twelve millimeters (12 mm) and no greater than one hundred millimeters (100 mm), such as no less than twenty-five millimeters (25 mm) and no greater than seventy-five millimeters (75 mm). As shown in FIG. 7, nonwoven fibrous web 210 may be unperforated between first and second feature zones 220, 230, e.g., along the longitudinal direction LO. Thus, e.g., the nonwoven fibrous web 210 may not be processed to include apertures between first and second feature zones 220, 230 along the longitudinal direction LO. In certain example embodiments, a centroid of first feature zone 220 may also be arranged colinear with a centroid of second feature zone 230, e.g., on the longitudinal direction LO. Moreover, first and second feature zones 220, 230 may be positioned at a center of nonwoven material, e.g., along the lateral direction LA. Thus, e.g., first and second feature zones 220, 230 may be positioned equidistant from first and second side portions 216, 218, e.g., along the lateral direction LA. Such placement of first and second feature zones 220, 230 within nonwoven material 200 may advantageously be consistently produced via method 10 due to reference line index 94 providing for registration of the modified substrate 14′ relative to the slitting device 15. Other suitable placements of first and second feature zones 220, 230 within nonwoven material 200 are also within the scope of the present subject matter and may be consistently produced via method 10 due to reference line index 94 providing for registration of the modified substrate 14′ relative to the slitting device 15.

First feature zone 220 may include a plurality of apertures 221 and a plurality of nodes 222, and second feature zone 230 may also include a plurality of apertures 231. Apertures 221 of first feature zone 220 may be larger than apertures 231 of second feature zone 230. For example, an area of each of apertures 221 of first feature zone 220 may be no less than about five millimeters squared (5 mm²) and no greater than about twenty-eight millimeters squared (28 mm²), such as no less than about eight millimeters squared (8 mm²) and no greater than about twenty millimeters squared (20 mm²), such as no less than about nine millimeters squared (9 mm²) and no greater than about twelve millimeters squared (12 mm²). In contrast, an area of each of apertures 231 of second feature zone 230 may be no less than about a quarter millimeter squared (0.25 mm²) and no greater than about five millimeters squared (5 mm²), such as no less than about one millimeter squared (1 mm²) and no greater than about four millimeters squared (4 mm²), such as about two and a half millimeters squared (2.5 mm²). Such sizing differential between apertures 221 of first feature zone 220 and apertures 231 of second feature zone 230 may advantageously facilitate movement of body exudates through nonwoven fibrous web 210. Moreover, the larger apertures 221 of first feature zone 220 may be sized for transferring and trapping low-viscosity fecal matter. Conversely, the smaller apertures 231 of second feature zone 230 may be sized for transferring and trapping urine exudate. In certain example embodiments, apertures 221 may be uniformly distributed throughout first feature zone 220, and apertures 231 may be uniformly distributed throughout second feature zone 230. The area of the apertures 221, 231 within the first and second features zones 220, 230 may be measured using the analysis techniques in the Material Sample Analysis Test Method as described in the Test Methods section herein.

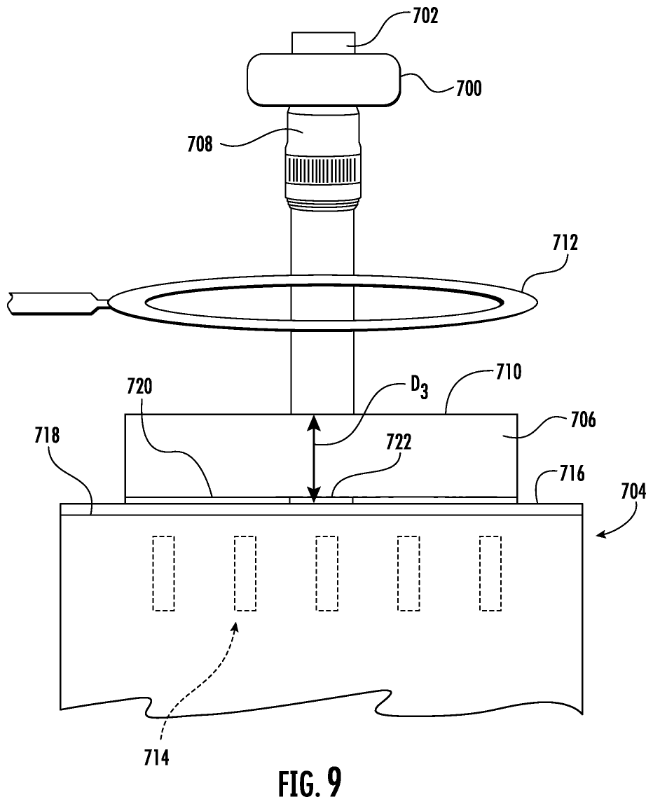

As noted above, nonwoven material 200 may include nodes 222 at first feature zone 220. It will be understood that nonwoven material 200 may include nodes at other locations on nonwoven material 200 in alternative example embodiments. Moreover, it will be understood that, in certain example embodiments, first feature zone 220 may include nodes 222 without apertures 221. As shown in FIG. 8, nonwoven fibrous web 210 may also include a plurality of connecting ligaments 223 at first feature zone 220. The nodes 222 may extend away from a base plane on a first surface of the nonwoven material 200. The base plane may be defined as the generally planar region of the first surface of the nonwoven material 200 other than the portion of the nonwoven material 200 forming the nodes 222. In other words, for the example embodiment depicted in FIGS. 8 and 9, the base plane may be formed by the first surface of the nonwoven material 200 that provides the connecting ligaments 223. The nonwoven material 200 may also include a second surface positioned opposite from the first surface on nonwoven fibrous web 210.

The nodes 222 may be configured in a variety of shapes and sizes. In some example embodiments, the nodes 222 may be generally cylindrical in shape. In certain example embodiments, the nodes 222 may be configured to not include any openings or apertures. In certain example embodiments, the nodes 222 may have a height (as measured in a direction perpendicular to the base plane of between about a half millimeter (0.5 mm) to about ten millimeters (10 mm), such as from about one millimeter (1 mm) to about three millimeters (3 mm), such as from about one and one-tenth millimeter (1.1 mm) to about one and five-tenths millimeters (1.5 mm), such as about one and three-tenths millimeters (1.3 mm). The height of the nodes 222 may be measured using the analysis techniques described in the Node Analysis Test Method described in the Test Methods section herein. In certain example embodiments, a majority of the nodes 222 may each have an area (as measured by the area of the node 222 at the base plane) from about five millimeters squared (5 mm²) to about thirty-five millimeters squared (35 mm²), such as from about ten millimeters squared (10 mm²) to about twenty millimeters squared (20 mm²). The nodes 222 may be configured in the first feature zone 220 such that the nodes 222 provide a node density of about one node per square centimeter (1.0 nodes/cm²) to about three nodes per square centimeter (3.0 nodes/cm²). The node area and node density within first feature zone 220 may be measured using the analysis techniques described in the Material Sample Analysis Test Method described in the Test Methods section herein.

As depicted in FIG. 7 and in more detail in FIG. 8, the connecting ligaments 223 may interconnect the nodes 222. Moreover, each connecting ligament 223 may be referred to as extending between only two (2) adjacent nodes 222. In other words, an individual, discrete connecting ligament 223 may not interconnect three (3) or more nodes 222. In example embodiments, a majority of nodes 222 may include at least three (3) connecting ligaments 223 connecting to adjacent nodes 222. In certain example embodiments, a majority of nodes 222 may include ten (10) or less connecting ligaments 223 connecting to adjacent nodes 222. In some example embodiments, the nonwoven material 200 may be configured such that a majority of nodes 222 may include three (3) to eight (8) connecting ligaments 223 connecting to adjacent nodes 222. For example, in the example embodiment in FIG. 7, a majority of the nodes 222 include six connecting ligaments 223 that connect to adjacent nodes 222.

The apertures 221 may be areas of the nonwoven fibrous web 210 that have a lower density of fibers of the nonwoven fibrous web 210 in comparison to nodes 222 and connecting ligaments 223. In some example embodiments, the apertures 221 may be substantially devoid of fibers. As used herein, the apertures 221 are to be distinguished from the normal interstitial fiber-to-fiber spacing commonly found in fibrous nonwoven materials. For example, the image in FIG. 8 shows that apertures 221 include a lower density of fibers than adjacent nodes 222 and connecting ligaments 223. The apertures 221 may be formed between the connecting ligaments 223 and the nodes 222. Individual apertures 221 may be disposed between adjacent nodes 222. In certain example embodiments, each aperture 221 may be defined between at least three (3) connecting ligaments 223 and at least three (3) nodes 222. In some example embodiments, individual, discrete apertures 221 may be defined between at least four (4) connecting ligaments 223 and at least four (4) nodes 222.

As may be seen from the above, the present subject matter may advantageously assist with slitting and spooling of nonwoven webs with discrete aperture zones. Moreover, the spacing or discontinuity between the discrete aperture zones may cause a visual registration system to lose a detection sequence and erratically control the web while searching for the next sequential aperture zone if the discrete aperture zones were used for registration. In contrast, the reference line index may be continuous and thus accurately guide and align the lanes of the discrete aperture zones during the slitting and spooling process. The visual registration system may detect the apertures in the reference line index and guide the web through the slitting knives.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

### Test Methods

### Material Sample Analysis Test Method

The Material Sample Analysis Test Method as described herein can be used for determining the size of an aperture 221, the roundness of an aperture 221, the aspect ratio for an aperture 221, the two-dimensional area of a node 222, and node 222 density and spacing. This test method involves obtaining two separate digital images of the sample.

### Material Apertured Zone Sample Analysis Set-Up and Determination

An exemplary setup for acquiring the images of the apertured zone is representatively illustrated in FIG. 9. Specifically, a CCD video camera 700 (e.g., a Leica DFC 300 FX video camera available from Leica Microsystems of Heerbrugg, Switzerland) is mounted on a standard support 702, such as a Polaroid MP-4 Land Camera standard support formerly available from Polaroid Resource Center in Cambridge, MS, and now potentially available from a resource such as eBay. The standard support 702 is attached to a macro-viewer 704, such as a KREONITE macro-viewer available from Dunning Photo Equipment, Inc., having an office in Bixby, Oklahoma. An auto stage 706 is placed on the upper surface of the macro-viewer 704. The auto stage 706 is used to automatically move the position of a given sample for viewing by the camera 700. A suitable auto stage 706 is Model H112, available from Prior Scientific Inc., having an office in Rockland, MA.

The specimen (not shown in FIG. 9) is placed on the auto stage 706 of a Leica Microsystems QWIN Pro Image Analysis system, under the optical axis of a sixty-millimeter (60 mm) lens 708 having an f-stop setting of four (4), such as a Nikon AF Micro Nikkor, manufactured by Nikon Corporation, having an office in Tokyo, Japan. The lens 708 is attached to the camera 700 using a c-mount adaptor. The distance from the front face of the lens 708 to the sample is approximately fifty-five centimeters (55 cm). The sample is laid flat on the auto stage surface 710 and any wrinkles removed by gentle stretching and/or fastening it to the auto stage surface 710 using transparent adhesive tape at outer edges of the sample. The sample surface is illuminated with incident fluorescent lighting provided by a sixteen inch (16″) diameter, forty watt (40 w), Circline fluorescent light 712, such as that manufactured by General Electric Company, having an office in Boston, MA. The light 712 is contained in a fixture that is positioned so the light 712 is centered over the sample and is approximately three centimeters (3 cm) above the sample surface. The illumination level of the light 712 is controlled with a Variable Auto-transformer (not shown), type 3PN1010, available from Staco Energy Products Co. having an office in Dayton, OH. Transmitted light is also provided to the sample from beneath the auto stage by a bank of four, two foot (2 ft.), EMC, Double-End Powered LED tube light 714 which are dimmable and available from Fulight Optoelectronic Materials, LLC. The LED light 714 are covered with a diffusing plate 716. The diffusing plate 716 is inset into, and forms a portion of, the upper surface 718 of the macro-viewer 704. This illumination source is overlaid with black mask 720 possessing a 3-inch by 3-inch opening 722. The opening 722 is positioned so that opening 722 is centered under the optical axis of the camera 700 and lens 708 system. The distance D₃ from the fluorescent light opening 722 to the surface 710 of the auto stage 706 is approximately seventeen centimeters (17 cm). The illumination level of the fluorescent light bank 714 is also controlled with a separate power control box (not shown) configured for dimmable LED lights.

The image analysis software platform used to perform the percent open area and aperture size measurements is a QWIN Pro (Version 3.5.1) available from Leica Microsystems, having an office in Heerbrugg, Switzerland. Alternatively, LAS Macro Editor, the next generation of software following QWIN Pro, could be used to perform the analysis. The system and images are also accurately calibrated using the QWIN software and a standard ruler with metric markings at least as small as one millimeter. The calibration is performed in the horizontal dimension of the video camera image. Units of millimeters per pixel are used for the calibration.

Thus, the method for determining the percent open area and opening size of a given specimen includes the step of performing measurements on the transmitted light image. Specifically, an image analysis algorithm is used to acquire and process images as well as perform measurements using Quantimet User Interactive Programming System (QUIPS) language. The image analysis algorithm is reproduced below. For purposes of clarity, the references in the algorithm to “bumps” or “projections” refers to nodes 222 for the nonwoven material 200 and the references to “open areas” or “apertures” refers to openings 221 for the nonwoven material 200.

DEFINE VARIABLES & OPEN FILES

The following line designates the computer location where data is sent to
Open File (C:\Data\94054 - Nhan (patent)\data.xls, channel #1)
TOTCOUNT = 0
TOTFIELDS = 0
MFRAMEH = 875
MFRAMEW = 1249
SAMPLE ID AND SET UP
Configure ( Image Store 1392 x 1040, Grey Images 81, Binaries 24 )
Enter Results Header
File Results Header ( channel #1 )
File Line ( channel #1 )
PauseText ( “Enter sample image prefix name now.” )
Input ( TITLE$ )
PauseText ( “Set sample into position.” )
Image Setup DC Twain [PAUSE] ( Camera 1, AutoExposure Off, Gain 0.00, ExposureTime 34.23
msec, Brightness 0, Lamp 38.83 )
Measure frame ( x 74, y 110, Width 1249, Height 875 )
Image frame ( x 0, y 0, Width 1392, Height 1040 )
-- Calvalue = 0.0377 mm/px
CALVALUE = 0.0377
Calibrate ( CALVALUE CALUNITS$ per pixel )
FRMAREA = MFRAMEH*MFRAMEW*(CALVALUE**2)
Clear Accepts
For ( SAMPLE = 1 to 1, step 1 )
Clear Accepts
File ( “Field No.”, channel #1, field width: 9, left justified )
File ( “% Open Area”, channel #1, field width: 7, left justified )
File ( “Bump Density”, channel #1, field width: 13, left justified )
File ( “Bump Spacing”, channel #1, field width: 15, left justified )
File Line ( channel #1 )
Stage ( Define Origin )
Stage ( Scan Pattern, 1 x 5 fields, size 82500.000000 x 39000.000000 )
IMAGE ACQUISITION I - Projection isolation
For ( FIELD = 1 to 5, step 1 )
Measure frame ( x 74, y 110, Width 1249, Height 875 )
Display ( Image0 (on), frames (on,on), planes (off,off,off,off,off,off), lut 0, x 0, y 0, z 1, Reduction off )
PauseText ( “Ensure incident lighting is correct (WL = 0.88 - 0.94) and acquire image.” )
Image Setup DC Twain [PAUSE] ( Camera 1, AutoExposure Off, Gain 0.00, ExposureTime 34.23
msec, Brightness 0, Lamp 38.83 )
Acquire ( into Image0 )
DETECT - Projections only
PauseText ( “Ensure that threshold is set at least to the right of the left gray-level histogram peak
which corresponds to the ‘land’ region.” )
Detect [PAUSE] ( whiter than 129, from Image0 into Binary0 delineated )
BINARY IMAGE PROCESSING
Binary Amend ( Close from Binary0 to Binary1, cycles 10, operator Disc, edge erode on )
Binary Identify ( FillHoles from Binary1 to Binary1 )
Binary Amend ( Open from Binary1 to Binary2, cycles 20, operator Disc, edge erode on )
Binary Amend ( Close from Binary2 to Binary3, cycles 8, operator Disc, edge erode on )
PauseText ( “Toggle <control> and <b> keys to check projection detection and correct if necessary.” )
Binary Edit [PAUSE] ( Reject from Binary3 to Binary3, nib Fill, width 2 )
Binary Logical ( copy Binary3, inverted to Binary4 )
IMAGE ACQUISITION 2 - % Open Area & Aperture Size
Measure frame ( x 74, y 110, Width 1249, Height 875 )
Display ( Image0 (on), frames (on,on), planes (off,off,off,off,off,off), lut 0, x 0, y 0, z 1, Reduction off )
PauseText ( “Turn off incident light & ensure transmitted lighting is correct (WL = 0.95) and acquire
image.” )
Image Setup DC Twain [PAUSE] ( Camera 1, AutoExposure Off, Gain 0.00, ExposureTime 34.23
msec, Brightness 0, Lamp 38.83 )
Acquire ( into Image0 )
ACQFILE$ = “C:\Images\94054 - Nhan\“+TITLE$+”_“+STR$(FIELD)+”.jpg”
Write image ( from ACQOUTPUT into file ACQFILE$ )
DETECT - Open areas only
Detect ( whiter than 127, from Image0 into Binary10 delineated )
BINARY IMAGE PROCESSING
Binary Amend ( Close from Binary10 to Binary11, cycles 5, operator Disc, edge erode on )
Binary Identify ( FillHoles from Binary11 to Binary12 )
Binary Amend ( Open from Binary12 to Binary13, cycles 10, operator Disc, edge erode on )
Binary Identify ( EdgeFeat from Binary13 to Binary14 )
PauseText ( “Ensure apertures are detected accurately.” )
Binary Edit [PAUSE] ( Reject from Binary14 to Binary14, nib Fill, width 2 )
FIELD MEASUREMENTS - % Open Area, Bump Density & Spacing
-- % open area
MFLDIMAGE = 10
Measure field ( plane MFLDIMAGE, into FLDRESULTS(1), statistics into FLDSTATS(7,1) )
Selected parameters: Area %
Field Histogram #1 ( Y Param Number, X Param Area %, from 0. to 60., linear, 20 bins )
PERCOPENAREA = FLDRESULTS(1)
-- bump density & spacing
MFLDIMAGE = 3
Measure field ( plane MFLDIMAGE, into FLDRESULTS(5), statistics into FLDSTATS(7,5) )
Selected parameters: Area, Intercept H, Intercept V, Area %,
Count/Area
BUMPDENSITY = FLDRESULTS(5)
MNSPACE1 = (FRMAREA-FLDRESULTS(1))/(FLDRESULTS(2)+FLDRESULTS(3))/2
Field Histogram #2 ( Y Param Number, X Param MNSPACE1, from 0. to 50., linear, 25 bins )
File ( FIELD, channel #1, 0 digits after ‘.’)
File ( PERCOPENAREA, channel #1, 1 digit after ‘.’)
File ( BUMPDENSITY, channel #1, 1 digit after ‘.’)
File ( MNSPACE1, channel #1, 1 digit after ‘.’)
File Line ( channel #1 )
FEATURE MEASUREMENTS - Aperture and bump sizes
-- Bump Size
Measure feature ( plane Binary3, 8 ferets, minimum area: 24, grey image: Image0 )
Selected parameters: Area, X FCP, Y FCP, EquivDiam
Feature Histogram #1 ( Y Param Number, X Param Area, from 1. to 100., logarithmic, 20 bins )
Feature Histogram #2 ( Y Param Number, X Param EquivDiam, from 1. to 100., logarithmic, 20 bins )
-- Aperture Size
Measure feature ( plane Binary14, 8 ferets, minimum area: 24, grey image: Image0 )
Selected parameters: Area, X FCP, Y FCP, Roundness, AspectRatio,
EquivDiam
Feature Histogram #3 ( Y Param Number, X Param Area, from 1. to 100., logarithmic, 20 bins )
Feature Histogram #4 ( Y Param Number, X Param EquivDiam, from 1. to 100., logarithmic, 20 bins )
Feature Histogram #5 ( Y Param Number, X Param Roundness, from 0.8999999762 to 2.900000095,
linear, 20 bins )
Feature Histogram #6 ( Y Param Number, X Param AspectRatio, from 1. to 3., linear, 20 bins )
Stage ( Step, Wait until stopped + 1100 msecs )
Next ( FIELD )
Next ( SAMPLE )
File Line ( channel #1 )
File Line ( channel #1 )
OUTPUT FEATURE HISTOGRAMS
File ( “Bump Size (area - sq. mm)”, channel #1 )
File Line ( channel #1 )
File Feature Histogram Results ( #1, differential, statistics, bin details, channel #1 )
File Line ( channel #1 )
File Line ( channel #1 )
File ( “Bump Size (ECD - mm)”, channel #1 )
File Line ( channel #1 )
File Feature Histogram Results ( #2, differential, statistics, bin details, channel #1 )
File Line ( channel #1 )
File Line ( channel #1 )
File ( “Aperture Size (area - sq. mm)”, channel #1 )
File Line ( channel #1 )
File Feature Histogram Results ( #3, differential, statistics, bin details, channel #1 )
File Line ( channel #1 )
File Line ( channel #1 )
File ( “Aperture Size (ECD - mm)”, channel #1 )
File Line ( channel #1 )
File Feature Histogram Results ( #4, differential, statistics, bin details, channel #1 )
File Line ( channel #1 )
File Line ( channel #1 )
File ( “Aperture Roundness”, channel #1 )
File Line ( channel #1 )
File Feature Histogram Results ( #5, differential, statistics, bin details, channel #1 )
File Line ( channel #1 )
File Line ( channel #1 )
File ( “Aperture Aspect Ratio”, channel #1 )
File Line ( channel #1 )
File Feature Histogram Results ( #6, differential, statistics, bin details, channel #1 )
File Line ( channel #1 )
File Line ( channel #1 )
Close File ( channel #1 )
END

The QUIPS algorithm is executed using the QWIN Pro software platform. The analyst is initially prompted to enter the specimen set information which is sent to the EXCEL file.

The analyst then enters an image file prefix name corresponding to the specimen identification. This will be used by the algorithm to save images acquired during the analysis to a specified file location. The analyst is next prompted by a live image set up window on the computer monitor screen to place a specimen onto the auto-stage. The sample should be laid flat and gentle force applied at edges of the sample to remove any macro-wrinkles that may be present. At this time, the Circline fluorescent light 712 can be on to assist in positioning the specimen. Next, the analyst is prompted to adjust the incident Circline fluorescent incident light 712 via the Variable Auto-transformer to a white level reading of approximately 0.9. The sub-stage transmitted light should either be turned off at this time or masked using a piece of light-blocking, black construction paper placed over the three (3) inch by three (3) inch opening 722.

The analyst is now prompted to ensure that the detection threshold is set to the proper level for detection of the nodes 222 using the Detection window which is displayed on the computer monitor screen. Typically, the threshold is set using the white mode at a point approximately near the middle of the 8-bit gray-level range (e.g., 127). If necessary, the threshold level can be adjusted up or down so that the resulting detected binary will optimally encompass the nodes 222 shown in the acquired image.

After the algorithm automatically performs several binary image processing steps on the detected binary of the nodes 222, the analyst will be given an opportunity to re-check node detection and correct any inaccuracies. The analyst can toggle both the ‘control’ and ‘b’ keys simultaneously to re-check node detection against the underlying acquired gray-scale image. If necessary, the analyst can select from a set of binary editing tools (e.g. draw, reject, etc.) to make any minor adjustments. If care is taken to ensure proper illumination and detection in the previously described steps, little or no correction at this point should be necessary.

Next, the analyst is prompted to turn off the incident Circline fluorescent light 712 and either turn on the sub-stage transmitted light or remove the light blocking mask. The sub-stage transmitted light is adjusted by the LED power controller to a white level reading of approximately 0.95. At this point, the image focus can be optimized for the apertured zone (such as first feature zone 220) of the material 200 including apertures 221.

The algorithm, after performing additional operations on the resulting separate binary images for apertures 221, will then prompt the analyst to re-check aperture 221 detection against the underlying gray-scale image. If necessary, the analyst can select from a set of binary editing tools (e.g. draw, reject, etc.) to make any minor adjustments.

The algorithm will then automatically perform measurements and output the data into a designated EXCEL spreadsheet file.

Following the transfer of data, the algorithm will direct the auto-stage to move to the next field-of-view and the process of turning on the incident, Circline fluorescent light 712 and blocking the transmitted sub-stage lighting will begin again. This process will repeat four times so that there will be five sets of data from five separate field-of-view images per single sampling replicate.

After completion of the analysis, the following measurement parameter data will be located in the EXCEL file after measurements and data transfer has occurred:
Percent Open Area
Node Density (No. per sq. metre)
Node Spacing (mm)
Node Size (One histogram for area in mm² and one histogram for equivalent circular diameter in mm)
Aperture Size (One histogram for area in mm² and one histogram for equivalent circular diameter in mm)
Aperture Roundness
Aperture Aspect Ratio

The final specimen mean spread value is usually based on an N=5 analysis from five, separate, specimen subsample replicates. A comparison of the percent open area, aperture 221 (aperture) size and other parameters acquired by the algorithm between different specimens can be performed using a Student's T analysis at the 90% confidence level.

### Example Embodiments

First example embodiment: A formation surface, comprising: a drum defining a plurality of lanes that are spaced apart along an axial direction on the drum, the plurality of lanes comprising a first lane and a second lane, the first lane comprising one or more discrete feature zones, the second lane comprising one or more discrete feature zones, wherein the drum further defines a reference line, the reference line comprising a plurality of features that are distributed around the drum along the circumferential direction, the features of the reference line spaced from the first lane along the axial direction.

Second example embodiment: The formation surface of the first example embodiment, wherein the features of the reference line are spaced from the first lane along the axial direction by no less than twenty millimeters and no greater than one hundred millimeters.

Third example embodiment: The formation surface of either the first example embodiment or the second example embodiment, wherein the features of the reference line are uniformly spaced along the circumferential direction on the drum.

Fourth example embodiment: The formation surface of any one of the first through third example embodiments, wherein each feature of the reference line is spaced from an adjacent feature of the reference line along the circumferential direction by no less than a half millimeter and no greater than ten millimeters.

Fifth example embodiment: The formation surface of any one of the first through fourth example embodiments, wherein the reference line is positioned at an end portion of the drum, the reference line disposed between the first lane and the end portion of the drum along the axial direction.

Sixth example embodiment: The formation surface of any one of the first through fifth example embodiments, wherein the features of the reference line comprise a plurality of pins extending from an outer surface of the drum along a radial direction.

Seventh example embodiment: The formation surface of the sixth example embodiment, wherein a smallest dimension of the pins is no less than one-tenth of a millimeter and no greater than ten millimeters.

Eighth example embodiment: The formation surface of any one of the first through seventh example embodiments, wherein the plurality of lanes comprises no less than four lanes.

Nineth example embodiment: A formation surface, comprising: a wall defining a plurality of lanes that are spaced apart along a lateral direction on the wall, the plurality of lanes comprising a first lane and a second lane, the first lane comprising one or more discrete feature zones that are spaced apart along a longitudinal direction on the wall, the second lane comprising one or more discrete feature zones that are spaced apart along the longitudinal direction on the wall, wherein the wall further defines a reference line, the reference line comprising a plurality of projections that are distributed on the wall along the longitudinal direction, the projections of the reference line spaced from the first lane along the lateral direction.

Tenth example embodiment: The formation surface of the nineth example embodiment, wherein the projections of the reference line are spaced from the first lane along the lateral direction by no less than twenty millimeters and no greater than one hundred millimeters.

Eleventh example embodiment: The formation surface of either the nineth example embodiment or the tenth example embodiment, wherein the projections of the reference line are uniformly spaced along the longitudinal direction on the wall.

Twelfth example embodiment: The formation surface of any one of the nineth through eleventh example embodiments, wherein each projection of the reference line is spaced from an adjacent projection of the reference line along the longitudinal direction by no less than a half millimeter and no greater than ten millimeters.

Thirteenth example embodiment: The formation surface of any one of the nineth through twelfth example embodiments, wherein the reference line is positioned at an end portion of the wall, the reference line disposed between the first lane and the end portion of the wall along the lateral direction.

Fourteenth example embodiment: The formation surface of any one of the nineth through thirteenth example embodiments, wherein the projections of the reference line comprise a plurality of pins extending outwardly from an outer surface of the wall.

Fifteenth example embodiment: The formation surface of the fourteenth example embodiment, wherein a smallest dimension of the pins is no less than one-tenth of a millimeter and no greater than ten millimeters.

Sixteenth example embodiment: The formation surface of one of the nineth through fifteenth example embodiments, wherein the plurality of lanes comprises no less than four lanes.

Seventeenth example embodiment: A formation system, comprising: the formation surface of any one of the nineth through sixteenth example embodiments, the formation surface configured to modify a substrate to include a plurality of discrete feature zone lanes corresponding to the plurality of lanes and to include a plurality of reference apertures corresponding to the reference line; a slitter configured to slit the substrate into a plurality of webs, each of the plurality of webs comprising a respective one or more of the plurality of discrete feature zone lanes; and a registration system configured for adjusting a position of the substrate relative to the slitter based at least in part upon images of the reference line.

Eighteenth example embodiment: A nonwoven material, comprising: a plurality of fibers forming a nonwoven fibrous web defining a lateral direction and a longitudinal direction, the lateral and longitudinal directions being perpendicular, the nonwoven fibrous web comprising a plurality of discrete feature zone lanes and a reference line, the plurality of discrete feature zone lanes spaced apart along the lateral direction, the plurality of discrete feature zone lanes comprising a first lane and a second lane, the first lane comprising one or more discrete feature zones, the second lane comprising one or more discrete feature zones, the reference line comprising a plurality of features that are distributed along the longitudinal direction, the features of the reference line spaced from the first lane along the lateral direction.

Nineteenth example embodiment: The nonwoven material of the eighteenth example embodiment, wherein the features of the reference line comprise apertures.

Twentieth example embodiment: The nonwoven material of either the eighteenth example embodiment or the nineteenth example embodiment, wherein features of the one or more feature zones comprise apertures.

Twenty-first example embodiment: The nonwoven material of any one of the eighteenth through twentieth example embodiments, wherein the features of the reference line comprise protrusions.

Twenty-second example embodiment: The nonwoven material of any one of the eighteenth through twenty-first example embodiments, wherein the features of the reference line and the one or more feature zones of the first lane and the second lane are integrally formed in the nonwoven material.

1. A formation surface, comprising:
a drum defining a plurality of lanes that are spaced apart along an axial direction on the drum, the plurality of lanes comprising a first lane and a second lane, the first lane comprising one or more discrete feature zones and one or more transition zones adjacent to the one or more discrete features zones, the second lane comprising one or more discrete feature zones and one or more transition zones adjacent to the one or more discrete features zones,
wherein the drum further defines a reference line, the reference line comprising a plurality of features that are distributed around the drum along a circumferential direction, the features of the reference line spaced from the first lane along the axial direction, wherein the reference line is adjacent to the one or more discrete feature zones and the one or more transition zones of the first lane and the second lane.

2. The formation surface of claim 1, wherein the features of the reference line are spaced from the first lane along the axial direction by no less than twenty millimeters and no greater than one hundred millimeters.

3. The formation surface of claim 1, wherein the features of the reference line are uniformly spaced along the circumferential direction on the drum.

4. The formation surface of claim 1, wherein each feature of the reference line is spaced from an adjacent feature of the reference line along the circumferential direction by no less than a half millimeter and no greater than ten millimeters.

5. The formation surface of claim 1, wherein the reference line is positioned at an end portion of the drum, the reference line disposed between the first lane and the end portion of the drum along the axial direction.

6. The formation surface of claim 1, wherein the features of the reference line comprise a plurality of pins extending from an outer surface of the drum along a radial direction.

7. The formation surface of claim 6, wherein a smallest dimension of the pins is no less than one-tenth of a millimeter and no greater than ten millimeters.

8. The formation surface of claim 1, wherein the plurality of lanes comprises no less than four lanes.

9. A formation surface, comprising:
a wall defining a plurality of lanes that are spaced apart along a lateral direction on the wall, the plurality of lanes comprising a first lane and a second lane, the first lane comprising one or more discrete feature zones that are spaced apart along a longitudinal direction on the wall and one or more transition zones adjacent to the one or more discrete features zones, the second lane comprising one or more discrete feature zones that are spaced apart along the longitudinal direction on the wall and one or more transition zones adjacent to the one or more discrete features zones,
wherein the wall further defines a reference line, the reference line comprising a plurality of projections that are distributed on the wall along the longitudinal direction, the projections of the reference line spaced from the first lane along the lateral direction, wherein the reference line is adjacent to the one or more discrete feature zones and the one or more transition zones of the first lane and the second lane.

10. The formation surface of claim 9, wherein the projections of the reference line are spaced from the first lane along the lateral direction by no less than twenty millimeters and no greater than one hundred millimeters.

11. The formation surface of claim 9, wherein the projections of the reference line are uniformly spaced along the longitudinal direction on the wall.

12. The formation surface of claim 9, wherein each projection of the reference line is spaced from an adjacent projection of the reference line along the longitudinal direction by no less than a half millimeter and no greater than ten millimeters.

13. The formation surface of claim 9, wherein the reference line is positioned at an end portion of the wall, the reference line disposed between the first lane and the end portion of the wall along the lateral direction.

14. The formation surface of claim 9, wherein the projections of the reference line comprise a plurality of pins extending outwardly from an outer surface of the wall.

15. The formation surface of claim 14, wherein a smallest dimension of the pins is no less than one-tenth of a millimeter and no greater than ten millimeters.

16. The formation surface of claim 9, wherein the plurality of lanes comprises no less than four lanes.

17. A formation system, comprising:
the formation surface of claim 9, the formation surface configured to modify a substrate to include a plurality of discrete feature zone lanes corresponding to the plurality of lanes and to include a plurality of reference features corresponding to the reference line;
a slitter configured to slit the substrate into a plurality of webs, each of the plurality of webs comprising a respective one or more of the plurality of discrete feature zone lanes; and
a registration system configured for adjusting a position of the substrate relative to the slitter based at least in part upon images of the reference line.

18. A nonwoven material, comprising:
a plurality of fibers forming a nonwoven fibrous web defining a lateral direction and a longitudinal direction, the lateral and longitudinal directions being perpendicular,
the nonwoven fibrous web comprising a plurality of discrete feature zone lanes and a reference line, the plurality of discrete feature zone lanes spaced apart along the lateral direction, the plurality of discrete feature zone lanes comprising a first lane and a second lane, the first lane comprising one or more discrete feature zones and one or more transition zones adjacent to the one or more discrete features zones, the second lane comprising one or more discrete feature zones and one or more transition zones adjacent to the one or more discrete features zones, the reference line comprising a plurality of features that are distributed along the longitudinal direction, the features of the reference line spaced from the first lane along the lateral direction, wherein the reference line is adjacent to the one or more discrete feature zones and the one or more transition zones of the first lane and the second lane.

19. The nonwoven material of claim 18, wherein the features of the reference line comprise apertures.

20. The nonwoven material of claim 18, wherein features of the one or more feature zones comprise apertures.

21. The nonwoven material of claim 18, wherein the features of the reference line comprise protrusions.

22. The nonwoven material of claim 18, wherein the features of the reference line and the one or more feature zones of the first lane and the second lane are integrally formed in the nonwoven material.
